(19) United States
(12) Patent Application Publication
(10) Pub. No.: US 2018/0264089 A1
(43) Pub. Date: Sep. 20, 2018

(54) MODIFIED IDURONATE 2-SULFATASE AND PRODUCTION THEREOF

(71) Applicant: SWEDISH ORPHAN BIOVITRUM AB (PUBL), Stockholm (SE)

(72) Inventors: Erik Nordling, Danderyd (SE); Patrick Strömberg, Sollentuna (SE); Stefan Svensson Gelius, Älvsjö (SE)

(21) Appl. No.: 15/764,194

(22) PCT Filed: Sep. 30, 2016
(86) PCT No.: PCT/EP2016/073476

§ 371 Date: Mar. 28, 2018

(30) Foreign Application Priority Data
Oct. 1, 2015 (EP) 15187871.7

(51) Int. Cl. A61K 38/46; C12N 9/16; A61P 3/00

(52) U.S. Cl. CPC A61K 38/46; C12Y 301/06013; A61P 3/00; C12N 9/16

(57) ABSTRACT

Disclosed herein are a modified iduronate 2-sulfatase, a composition comprising a modified iduronate 2-sulfatase, as well as methods for preparing a modified iduronate 2-sulfatase and therapeutic use of such a iduronate 2-sulfatase. In particular, the present disclosure relates to a modified iduronate 2-sulfatase sulfatase comprising substantially no epitopes for glycan recognition receptors, wherein said iduronate 2-sulfatase has a catalytic activity of at least 50% of that of unmodified iduronate 2-sulfatase in vitro.

### TECHNICAL FIELD

The present disclosure relates to a modified iduronate 2-sulfatase, compositions comprising a modified iduronate 2-sulfatase and methods for producing a modified iduronate 2-sulfatase. Furthermore, use of a modified iduronate 2-sulfatase in therapy such as in treatment of a lysosomal storage disease, as well as a method of treating a mammal afflicted with a lysosomal storage disease, is disclosed.

### BACKGROUND

### Lysosomal Storage Disease

The lysosomal compartment functions as a catabolic machinery that degrades waste material in cells. Degradation is achieved by a number of hydrolases and transporters compartmentalized specifically to the lysosome. There are today over 40 identified inherited diseases where a link has been established between disease and mutations in genes coding for lysosomal proteins. These diseases are defined as lysosomal storage diseases (LSDs) and are characterized by a buildup of a metabolite (or metabolites) that cannot be degraded due to the insufficient degrading capacity. As a consequence of the excess lysosomal storage of the metabolite, lysosomes increase in size. How the accumulated storage material cause pathology is not fully understood but may involve mechanisms such as inhibition of autophagy and induction of cell apoptosis (Cox & Cachón-Gonzalez, J Pathol 226: 241-254 (2012)).

### Enzyme Replacement Therapy

Storage can be reduced by administration of a lysosomal enzyme from a heterologous source. It is well established that intravenous administration of a lysosomal enzyme results in its rapid uptake by cells via a mechanism called receptor mediated endocytosis. This endocytosis is mediated by receptors on the cell surface, and in particular the two mannose-6 phosphate receptors (M6PR) have been shown to be pivotal for uptake of certain lysosomal enzymes (Neufeld; Birth Defects Orig Artic Ser 16: 77-84 (1980)). M6PR recognize phosphorylated oligomannose glycans which are characteristic for lysosomal proteins.

Based on the principle of receptor mediated endocytosis, enzyme replacement therapies (ERT) are today available for seven LSDs, (Gaucher, Fabrys, Pompe and the Mucopolysaccharidosis type I, II, IVA and VI). These therapies are efficacious in reducing lysosomal storage in various peripheral organs and thereby ameliorate some symptoms related to the pathology.

Elaprase® is an orphan medicinal product indicated for long-term treatment of patients with Hunter syndrome (Mucopolysaccharidosis II, MPSII), which is a rare X-linked recessive storage disorder caused by a deficiency or reduced levels of the lysosomal enzyme iduronate-2-sulfatase (I2S). This enzyme is responsible for the hydrolysis of the C2-sulfate ester bonds of the non-reducing iduronic acid residue in both glycosaminoglycans (GAGs) dermatan sulfate and heparan sulfate. Reduced or absent activity of this enzyme results in an intracellular accumulation of these GAGs, which causes a progressive and clinically heterogeneous disorder with multiple organ and tissue involvement.

However, a majority of the LSDs, including MPSII, causes build-up of lysosomal storage in the central nervous system (CNS) and consequently present a repertoire of CNS related signs and symptoms. A major drawback with intravenously administered ERT is the poor distribution to the CNS. The CNS is protected from exposure to blood borne compounds by the blood brain barrier (BBB), formed by the CNS endothelium. The endothelial cells of the BBB exhibit tight junctions which prevent paracellular passage, show limited passive endocytosis and in addition lack some of the receptor mediated transcytotic capacity seen in other tissues. Notably, in mice M6PR mediated transport across the BBB is only observed up to two weeks after birth (Urayama et al, Mol Ther 16: 1261-1266 (2008)).

In addition to the neurological component of LSDs, such as MPSII, peripheral pathology is to some extent also sub-optimally addressed in current enzyme replacement treatment. Patients frequently suffer from arthropathy, clinically manifested in joint pain and stiffness resulting in severe restriction of motion. Moreover, progressive changes in the thoracic skeleton may cause respiratory restriction.

Prevailing storage leading to thickening of the heart valves along with the walls of the heart can moreover result in progressive decline in cardiac function. Also pulmonary function can further regress despite enzyme replacement treatment.

### Glycosylation of Lysomal Enzymes

In general, N-glycosylations can occur at a Asn-X-Ser/Thr sequence motif. To this motif the initial core structure of the N-glycan is transferred by the glycosyltransferase oligosaccharyltransferase, within the reticular lumen. This common basis for all N-linked glycans is made up of 14 residues; 3 glucose, 9 mannose, and 2 N-acetylglucosamine. This precursor is then converted into three general types of N-glycans; oligomannose, complex and hybrid (FIG. 7), by the actions of a multitude of enzymes that both trim down the inital core and adds new sugar moieties. Each mature N-glycan contains the common core Man(Man)2-GlcNAc-GlcNAc-Asn, where Asn represents the attachment point to the protein. In yeast, oligomannose glycans can be extended to contain up to 200 mannose moieties in a repetitive fashion depicted at the far right in FIG. 7 (Dean, Biochimica et Biophysica Acta 1426:309-322 (1999)).

In addition, proteins directed to the lysosome carry one or more N-glycans which are phosphorylated. The phosphorylation occurs in the Golgi and is initiated by the addition of N-acetylglucosamine-1-phosphate to C-6 of mannose residues of oligomannose type N-glycans. The N-acetylglucosamine is cleaved off to generate Mannose-6-phospate (M6P) residues, that are recognized by M6PRs and will initiate the transport of the lysosomal protein to the lysosome. The resulting N-glycan is then trimmed to the point where the M6P is the terminal group of the N-glycan chain. (Essentials of Glycobiology. 2nd edition. Varki A, Cummings R D, Esko J D, et al, editors. Cold Spring Harbor (N.Y.): Cold Spring Harbor Laboratory Press; 2009.)

The binding site of the M6PR requires a terminal M6P group that is complete, as both the sugar moiety and the phosphate group is involved in the binding to the receptor (Kim et al, Curr Opin Struct Biol 19:534-42 (2009)).

### Enzyme Replacement Therapy Targeting the Brain by Glycan Modification

A potential strategy to increase distribution of lysomal enzyme to the CNS has been disclosed in e.g. WO 2008/109677 and US 2014/377246. In these publications, chemical modification of β-glucuronidase using sodium meta-periodate and sodium borohydride is described (see also Grubb et al, Proc Natl Acad Sci USA 105: 2616-2621 (2008)). This modification, consisting of oxidation with 20 mM sodium periodate for 6.5 h, followed by quenching, dialysis and reduction with 100 mM sodium borohydride overnight (referred to hereinafter as known method), substantially improved CNS distribution of R-glucuronidase and resulted in clearance of neuronal storage in a murine model of the LSD mucopolysaccharidosis VII. Although the underlying mechanism of brain distribution is unclear, it was noted that the chemical modification disrupted glycan structure on β-glucuronidase and it was further demonstrated that receptor mediated endocytosis by M6PR was strongly reduced.

The chemical modification strategy has been investigated for other lysosomal enzymes. For example, modification according to the known method did not improve distribution to the brain of intravenously administrated protease tripeptidyl peptidase I (Meng et al, PLoS One 7: e40509 (2012)). Neither has satisfactory results been demonstrated for sulfamidase. Sulfamidase, chemically modified according to the known method, did indeed display an increased half-life in mice but no effect in the brain of MPS-IIIIA mice. The chemically modified sulfamidase did not distribute to the brain parenchyma when given repeatedly by intravenous administration (Rozaklis et al, Exp Neurol 230: 123-130 (2011)).

Thus, there is still no effective intravenously administrated ERT for LSDs with neurological engagement, such as MPS-II. Novel iduronate 2-sulfatase polypeptides that can be transported across the BBB while remaining enzymatically active would be of great value in the development of systemically administrated compounds for enzyme replacement therapies for the treatment of LSDs with CNS related pathology, such as MPS-II.

### DISCLOSURE OF THE INVENTION

It is an object of the present invention to provide novel iduronate 2-sulfatase polypeptides allowing development of an enzyme replacement therapy for LSDs such as MPS-II.

It is another object of the present invention to provide a novel iduronate 2-sulfatase polypeptide that may be transported across the blood brain barrier in mammals and which may exhibit an enzymatic (catalytic) activity in the brain of said mammal.

Yet another object of the present invention is to provide a novel iduronate 2-sulfatase polypeptide that has catalytic activity in peripheral tissue, such as joints, bone, connective tissue and/or cartilage.

Yet another object of the present invention is to provide a novel iduronate 2-sulfatase polypeptide exhibiting improved stability, such as improved structural integrity.

These and other objects, which will be apparent to a skilled person from the present disclosure, are achieved by the different aspects of the invention as defined in the appended claims and as generally disclosed herein.

There is, in one aspect of the invention, provided a modified iduronate 2-sulfatase comprising substantially no epitopes for glycan recognition receptors, wherein said iduronate 2-sulfatase has a catalytic activity of at least 50% of that of unmodified iduronate 2-sulfatase in vitro, such as at least 60% of that of unmodified iduronate 2-sulfatase in vitro, such as at least 70% of that of unmodified iduronate 2-sulfatase in vitro, such as at least 80% of that of unmodified iduronate 2-sulfatase in vitro. The modified iduronate 2-sulfatase according to the invention may allow for a more effective therapy for mucopolysaccharidosis II (MPS-II). A method for measuring catalytic activity in vitro and a modified iduronate 2-sulfatase having at least 50% activity is disclosed in Example 2 and 4. The appended Examples moreover demonstrates that iduronate 2-sulfatase modified according to previously known methods has a catatytic activity in vitro of below 50% of that of unmodified iduronate 2-sulfatase. The present invention thus advantageously provides an improved modified iduronate 2-sulfatase in terms of catalytic activity.

The modified iduronate 2-sulfatase according to the invention is thus modified in that epitopes for glycan recognition receptors have been removed, for example as compared to an unmodified iduronate 2-sulfatase (SEQ ID NO:1). Such a modified iduronate 2-sulfatase is less prone to cellular uptake, as demonstrated in the cellular uptake studies of Example 5, which is a consequence of removal of epitopes for glycan recognition receptors such as the two mannose-6 phosphate receptors (M6PR). This might reduce the affinity of the modified iduronate 2-sulfatase with respect to glycan recognition receptors, and in particular the receptor mediated endocytosis of the modified iduronate 2-sulfatase in peripheral tissue. In this context, cellular uptake in peripheral tissue such as the liver may be reduced. In turn, this may result in a reduced clearance of modified iduronate 2-sulfatase from plasma when e.g. administrated intravenously to a mammal. From a dosing perspective, reduced clearance of modified iduronate 2-sulfatase may advantageously allow for development of long-acting medicaments that can be administered to patients less frequently.

By glycan recognition receptors is meant receptors that recognize and bind proteins mainly via glycan moieties of the proteins. Such receptors can, in addition to the mannose 6-phosphate receptors, be exemplified by the mannose receptor, which selectively binds proteins where glycans exhibit exposed terminal mannose residues. Lectins constitute another large family of glycan recognition receptors which can be exemplified by the terminal galactose recognizing asialoglycoprotein receptor 1 recognizing terminal galactose residues on glycans. Epitopes for glycan recognition receptors can thus be understood as (part of) glycan moieties recognized by such receptors.

In this context, a modified iduronate 2-sulfatase comprising substantially no epitopes for glycan recognition receptors should preferably be understood as a modified iduronate 2-sulfatase comprising nearly no epitopes for glycan recognition receptors, or only trace amounts of such epitopes. In preferred embodiments, the modified iduronate 2-sulfatase comprises no (detectable) epitopes for glycan recognition receptors. In particular, the modified iduronate 2-sulfatase comprises no (detectable) mannose-6-phosphate moieties, mannose moieties, N-acetylglucosamine moieties or galactose moieties that constitute epitopes for the endocytic M6PR type 1 and 2, the mannose receptor, lectins binding n-acetylglucosamine and the galactose receptor, respectively. Said epitopes, which are substantially absent in said modified iduronate 2-sulfatase, may, when present on unmodified iduronate 2-sulfatase, be recognized by glycan recognition receptors selected from mannose-6 phosphate receptor type 1 and 2, mannose receptor and galactose receptor. Mannose-6-phosphate moieties, mannose moieties and galactose moieties may represent said epitopes, which are found on natural glycan moieties of unmodified iduronate 2-sulfatase. In particular embodiments, these are absent from the modified iduronate 2-sulfatase as disclosed herein.

In one embodiment, said iduronate 2-sulfatase has catalytic activity in the brain of said mammal. The modified iduronate 2-sulfatase according to aspects described herein may not only be distributed to the brain of a mammal, but may also display (retained) enzymatic activity or catalytic activity in the brain of said mammal. By this is meant that the enzymatic activity of the modified iduronate 2-sulfatase is retained at least partly as compared to an unmodified form of the iduronate 2-sulfatase. Thus, the modified iduronate 2-sulfatase as disclosed herein may affect lysosomal storage in the brain of mammals, such as to decrease lysosomal storage, for example lysosomal storage of dermatan sulfate, heparan sulfate and heparin. The retained catalytic activity may for instance depend on level of preservation versus modification of a catalytic amino acid residue at the active site of iduronate 2-sulfatase.

In one embodiment, said iduronate 2-sulfatase has catalytic activity in peripheral tissue. Typically, said peripheral tissue may in this context be understood as peripheral tissue to which unmodified iduronate 2-sulfatase is poorly distributed and/or where lysosomal storage needs to be reduced. Thus, such peripheral tissue is for example joints, bone, connective tissue, skeletal muscle, heart, lung and/or cartilage. In particular, such peripheral tissue is joints, bone, connective tissue and/or cartilage. The distribution of modified iduronate 2-sulfatase as disclosed herein may be significantly enhanced to such peripheral tissues where unmodified iduronate 2-sulfatase typically is poorly distributed. In particular, the modified iduronate 2-sulfatase may display higher exposure in joints, connective tissue, cartilage and bone, when administrated by intravenous infusion. The modified iduronate 2-sulfatase may moreover display better distribution to skeletal muscle, heart and/or lung.

Iduronate 2-sulfatase belong to the protein family of sulfatases. Sulfatases are a family of proteins of common evolutionary origin that catalyze the hydrolysis of sulfate ester bonds from a variety of substrates. Thus, “catalytic activity” of modified iduronate 2-sulfatase as used herein may refer to hydrolysis of sulfate ester bonds, preferably in lysosomes of peripheral tissue and/or in lysosomes in the brain of a mammal. Catalytic activity of modified iduronate 2-sulfatase may thus result in reduction of lysosomal storage, such as storage of GAGs, e.g. dermatan sulfate and heparan sulfate, in the brain or in peripheral tissue of a mammal suffering from a lysosomal storage disease. Catalytic activity can be measured in an animal model, for example as described in Example 7.

Sulfatases share a common fold with a central β-sheet which consists of 10 β-strands. The active site of iduronate 2-sulfatase is located at the end of the central β-sheet and contains a conserved cysteine in position 59 of SEQ ID NO:1 that is post-translationally modified to a Ca-formylglycine (FGly). This reaction takes place in the endoplasmic reticulum by the FGly generating enzyme. This FGly residue in position 59 (FGly59) is directly involved in the hydrolysis of sulfate ester bonds and the modification seems necessary for the enzyme to be active. Notably, mutation of the conserved cysteine to a serine (Ser) in arylsulfatase A and B prevents FGly formation and yields inactive enzymes (Recksiek et al, J Biol Chem 13; 273(11):6096-103 (1998)). Glycan modification of sulfamidase, which is a related sulfatase, has been disclosed in the prior art (Rozaklis et al, supra). The known method for modifying sulfamidase however results in a modified sulfamidase lacking catalytic activity in the brain of mice. Thus, this shows that modification of an enzyme has to be carefully carried out in order not to jeopardize catalytic activity. In the case of iduronate 2-sulfatase, modification has to be conducted without causing conversion of FGly59 to Ser59, which would render the modified iduronate 2-sulfatase inactive. Thus, when preservation of active site is discussed herein, it should primarily be understood as preservation of the post-translational FGly59 of SEQ ID NO:1. In such instances the modified iduronate 2-sulfatase should be understood as comprising a polypeptide consisting of an amino acid sequence as defined in SEQ ID NO:1 or an amino acid sequence having a sequence identity as defined below with such an amino acid sequence.

In one embodiment, said active site comprises a catalytic residue in a position corresponding to position 59 of SEQ ID NO:1 providing said catalytic activity. This catalytic residue is in a further embodiment FGly59.

Human iduronate 2-sulfatase (EC:3.1.6.13, SEQ ID NO:1) is encoded by the IDS gene. The mature protein consists of 525 amino acids and has a molecular weight of approximately 76 kDa. Iduronate 2-sulfatase is also known under the names alpha-L-iduronate sulfate sulfatase and idursulfase (INN name). The term “iduronate 2-sulfatase” as used herein should be understood as being synonymous to these alternative names.

Iduronate 2-sulfatase contains two disulfide bonds and eight N-linked glycosylation sites occupied by complex, hybrid and high mannose type oligosaccharide chains. In one embodiment, the modified iduronate 2-sulfatase has a relative content of natural glycan moieties being around 38 or less of the content of natural glycan moieties in unmodified recombinant iduronate 2-sulfatase. Said epitopes for glycan recognition receptors may thus be found on natural glycan moieties, and such natural glycan moieties are thus substantially absent in the modified iduronate 2-sulfatase as described herein. Natural glycan moieties should in this respect be understood as glycan moieties naturally occurring in iduronate 2-sulfatase that are post-translationally modified in the endoplasmatic reticulum and golgi compartments of eukaryotic cells. The relative content of glycan moieties can be understood as the content of intact natural glycan moieties. As demonstrated in the appended Examples, relative quantification of glycopeptides may be based on LC-MS and peak areas from reconstructed ion chromatograms. Alternative quantification methods are known to the person skilled in the art. A relative content of natural glycans at a level of less than 38% may advantageously reduce receptor mediated endocytosis of iduronate 2-sulfatase into cells via glycan recognition receptors, and improve transportation across the blood brain barrier.

The relative content of natural glycan epitopes in modified iduronate 2-sulfatase may in preferred embodiments be less than 38%, such as less than 25%, such as less than 13%, such as less than 10%, such as less than 5%. In a particular embodiment, the content of natural glycan epitopes is less than 1%.

Said natural glycan moieties of the modified iduronate 2-sulfatase may be absent on the modified iduronate 2-sulfatase as accounted for above. This absence may correspond to disruption, consisting of single bond breaks and double bond breaks, within the natural glycan moieties in said modified iduronate 2-sulfatase. Glycan disruption by single bond break may typically be predominant. In particular, natural glycan moieties of said iduronate 2-sulfatase may be disrupted by single bond breaks and double bond breaks, wherein the extent of single bond breaks may be at least 60% in oligomannose glycans. In particular, the extent of single bond breaks may be at least 65%, such as at least 70%, such as at least 75%, such as at least 80%, such as at least 82%, such as at least 85% in the oliogomannose type of glycans. The extent of single bond breaks vs. double bond breaks may be determined as described in Examples 10 and 11. In one embodiment, said iduronate 2-sulfatase has molecular weight of more than 95% of that of unmodified iduronate 2-sulfatase, such as more than 96% of that of unmodified iduronate 2-sulfatase, such as more than 97% of that of unmodified iduronate 2-sulfatase, such as more than 98% of that of unmodified iduronate 2-sulfatase, such as more than 99% of that of unmodified iduronate 2-sulfatase. In appended Example 4 it is shown that the modified iduronate 2-sulfatase according to the invention is undistinguishable from the unmodified iduronate 2-sulfatase in an SDS-PAGE analysis, suggesting mainly single bond breaks, which is depicted in FIG. 8A. In appended Example 2 it is shown that the modified iduronate 2-sulfatase according to the known method is smaller than the unmodified iduronate 2-sulfatase in an SDS-PAGE analysis, suggesting a higher extent of double bond breaks, which is depicted in FIG. 8A.

In one embodiment, the modified iduronate 2-sulfatase comprises a polypeptide consisting of an amino acid sequence as defined in SEQ ID NO:1, or a polypeptide having at least 90% sequence identity with an amino acid sequence as defined in SEQ ID NO:1. In a non-limiting example, said polypeptide has at least 95% sequence identity with an amino acid sequence as defined in SEQ ID NO:1, such as at least 98% sequence identity with an amino acid sequence as defined in SEQ ID NO:1, such as at least 99 sequence identity with an amino acid sequence as defined in SEQ ID NO:1. The modified iduronate 2-sulfatase according to the invention may thus comprise a polypeptide having an amino acid sequence which is highly similar to the sequence of SEQ ID NO:1. Said polypeptide may however for example be extended by one or more C- and/or N-terminal amino acid(s), making the actual modified iduronate 2-sulfatase sequence longer than the sequence of SEQ ID NO:1. Similarly, in other instances the modified iduronate 2-sulfatase may have an amino acid sequence which is shorter than the amino acid sequence of SEQ ID NO:1, the difference in length e.g. being due to deletion(s) of amino acid residue(s) in certain position(s) of the sequence.

In one embodiment, said epitopes are absent at at least five of the eight N-glycosylation sites: asparagine (N) in position 6 (N(6)), asparagine (N) in position 90 (N(90)), N in position 119 (N(119)), N in position 221 (N(221)), N in position 255 (N(255)), N in position 300 (N(300)), N in position 488 (N(488)) and N in position 512 (N(512)) of SEQ ID NO:1. Thus, said modified iduronate 2-sulfatase has intact natural glycan moieties at no more than three of said N-glycosylation sites. Advantages of such a modified iduronate 2-sulfatase lacking intact or complete glycan moieties at the identified sites are as accounted for above; i.e. the cellular uptake might be further reduced and the transportation across the blood brain barrier might be further facilitated.

In one embodiment, said epitopes are absent at at least six of the eight N-glycosylation sites: asparagine (N) in position 6 (N(6)), asparagine (N) in position 90 (N(90)), N in position 119 (N(119)), N in position 221 (N(221)), N in position 255 (N(255)), N in position 300 (N(300)), N in position 488 (N(488)) and N in position 512 (N(512)) of SEQ ID NO:1. In one embodiment, the epitope in the glycosylation site asparagine (N) in position 90 (N(90)) is absent. In one embodiment, said epitopes are absent at at least seven of said eight N-glycosylation sites. In a particular embodiment, said epitopes are absent at all of said eight N-glycosylation sites. A modified iduronate 2-sulfatase lacking said epitopes may display further improved pharmacokinetics, for example in that the plasma clearance in a mammal may be further reduced. As a consequence, dosing frequency of a modified iduronate 2-sulfatase may hence also be further reduced.

In one embodiment of the aspects disclosed herein, said modified iduronate 2-sulfatase is present in a non-covalently linked form. Advantageously, said iduronate 2-sulfatase has been modified without causing aggregation of the protein and/or without causing cleavage of the protein backbone into smaller peptide fragments.

In one embodiment, said modified iduronate 2-sulfatase is isolated.

In one embodiment, said iduronate 2-sulfatase is human iduronate 2-sulfatase.

In one embodiment, said iduronate 2-sulfatase prior to modification is glycosylated.

In one embodiment, said modified iduronate 2-sulfatase is recombinant. In particular, iduronate 2-sulfatase may be recombinantly produced in a continuous human cell line. Iduronate 2-sulfatase may be recombinantly produced as described in Bielicki et al., Biochem J., 289: 241-246 (1993).

In another embodiment, said iduronate 2-sulfatase has been produced recombinantly in mammalian, plant or yeast cells. One example of a cell line is a CHO cell line. The resulting iduronate 2-sulfatase is thus, prior to modification, glycosylated by one or more oligomannose N-glycans.

In one embodiment of the invention, there is provided an iduronate 2-sulfatase composition, comprising modified iduronate 2-sulfatase as disclosed above, said composition having a Ca-formylglycine (FGly) to serine (Ser) ratio at the active site that is greater than 1.

In one aspect of the invention there is provided a modified iduronate 2-sulfatase comprising substantially no epitopes for glycan recognition receptors, thereby enabling transportation of said iduronate 2-sulfatase across the blood brain barrier of a mammal, wherein said iduronate 2-sulfatase has catalytic activity in the brain of said mammal. Embodiments of this aspect are disclosed above.

In one aspect, there is provided an iduronate 2-sulfatase composition, comprising modified iduronate 2-sulfatase having substantially no epitopes for glycan recognition receptors, thereby enabling transportation of said iduronate 2-sulfatase across the blood brain barrier of a mammal, and a Ca-formylglycine (FGly) to serine (Ser) ratio at the active site that is greater than 1, thereby providing catalytic activity in the brain of a mammal. For example, said modified iduronate 2-sulfatase comprises a polypeptide consisting of an amino acid sequence as defined in SEQ ID NO:1, or a polypeptide having at least 90% sequence identity with a polypeptide as defined in SEQ ID NO:1. In such examples, the FGly to Ser ratio may be referred to as a FGly59 to Ser59 ratio. Preferably, the ratio is larger than 1.5, more preferably larger than 2.3, more preferably larger than 4, and most preferably the ratio is around 9. A larger ratio indicates that the catalytic activity of the modified iduronate 2-sulfatase to a larger extent may be retained from an unmodified form of iduronate 2-sulfatase.

The advantages of a composition comprising a modified iduronate 2-sulfatase are similar to the advantages of a modified iduronate 2-sulfatase as such. Thus, a composition comprising modified iduronate 2-sulfatase may exhibit an improved half-life in plasma compared to an unmodified iduronate 2-sulfatase or a composition comprising unmodified iduronate 2-sulfatase. In addition, said modified iduronate 2-sulfatase may exhibit improved distribution to the brain of a mammal, as well as a retained catalytic activity in the brain, compared for example to an unmodified iduronate 2-sulfatase.

In one embodiment, the iduronate 2-sulfatase composition has a relative content of natural glycan moieties being around 38%, or less, of the content of natural glycan moieties in a composition of unmodified recombinant iduronate 2-sulfatase. Said epitopes for glycan recognition receptors may be found on natural glycan moieties, and such natural glycan moieties are thus substantially absent in the modified iduronate 2-sulfatase as described herein. A relative content of natural glycans at a level of around or less than 38 may advantageously reduce receptor mediated endocytosis of iduronate 2-sulfatase into cells via glycan recognition receptors, and improve transportation across the blood brain barrier. The relative content of natural glycan epitopes in the iduronate 2-sulfatase composition may in preferred embodiments be less than 25%, less than 13%, less than 10%, less than 5%. In some instances, the relative content of natural glycan moieties is less than 4%, 3%, 2%, 1%, 0.5%, such as less than 0.1%, such as less than 0.01%. In a particular embodiment, the content of natural glycan moieties is less than 1%. The relative content of glycan moieties can be understood as the content of intact natural glycan moieties.

In one particular embodiment of the composition aspect, said epitopes are absent at at least five of said eight N-glycosylation sites asparagine (N) in position 6 (N(6)), asparagine (N) in position 90 (N(90)), N in position 119 (N(119)), N in position 221 (N(221)), N in position 255 (N(255)), N in position 300 (N(300)), N in position 488 (N(488)) and N in position 512 (N(512)) of SEQ ID NO:1. Preferably said epitopes are absent at at least six of said eight N-glycosylation sites, such as at least seven of said N-glycosylation sites, such as all of said N-glycosylation sites.

In one embodiment of the composition aspect, no more than 10%, such as no more than 5% (by weight) of said modified iduronate 2-sulfatase is present in multimeric forms having a molecular weight of above 10¹⁰ kDa.

In one embodiment of the composition aspect, no more than 10%, such as no more than 5% (by weight) of said modified iduronate 2-sulfatase is present in covalently linked oligomeric forms. Said oligomeric forms being selected from dimers, trimers, tetramers, pentamers, hexamers, heptamers and octamers, or said oligomeric forms having a molecular weight of between 180 and 480 kDa. The presence of oligomeric, multimeric, or aggregated forms, can for example be determined by dynamic light scattering or by size exclusion chromatography. In this context, aggregated forms should be understood as high molecular weight protein forms composed of structures ranging from natively folded to unfolded monomers. Aggregated forms of a protein can enhance immune response to the monomeric form of the protein. The most likely explanation for an enhanced immune response is that the multivalent presentations of antigen cross link B-cell receptors and thus induce an immune response. This is a phenomenon which has been utilized in vaccine production where the antigen is presented to the host in an aggregated form to ensure a high immune response. For therapeutic proteins the dogma is the opposite; any content of high molecular weight forms should be minimized or avoided in order to minimize the immune response (Rosenberg, AAPS J, 8:E501-7 (2006)). Thus, reduction of oligomeric, multimeric and/or aggregate forms may thus provide an enzyme more suitable for use in therapy.

Moreover, the occurrence of even a small amount of aggregates in a protein composition may induce further aggregation of normally folded proteins. The aggregated material generally has no or low remaining activity and poor solubility. The appearance of aggregates can be one of the factors that determine the shelf-life of a biological medicine (Wang, Int J Pharm, 185:129-88 (1999)).

The term “composition” as used herein should be understood as encompassing solid and liquid forms. A composition may preferably be a pharmaceutical composition, suitable for administration to a patient (e.g. a mammal) for example by injection or orally.

It should moreover be understood that the embodiments and the advantages disclosed in relation to the modified iduronate 2-sulfatase aspects are embodiments also of the composition aspect. In the same way, the embodiments of the composition aspect should also be regarded as embodiments of the modified iduronate 2-sulfatase aspects, where applicable.

In one embodiment of the aspects disclosed herein, said modified iduronate 2-sulfatase or said iduronate 2-sulfatase composition is for use in therapy.

In one embodiment, said mammalian brain is the brain of a human being. In a related embodiment, said mammal is thus a human.

In one embodiment, said mammalian brain is the brain of a mouse. In a related embodiment, said mammal is thus a mouse.

In one embodiment, said modified iduronate 2-sulfatase or iduronate 2-sulfatase composition is for use in treatment of a mammal afflicted with a lysomal storage disease, in particular mucopolysaccharidosis II (MPS-II; Hunter syndrome).

In one embodiment, said modified iduronate 2-sulfatase or iduronate 2-sulfatase composition for use reduces GAG storage in the brain of said mammal. In particular, storage of heparan sulfate storage and/or dermatan sulfate may be reduced. In certain instances, said heparan sulfate storage and/or dermatan sulfate is reduced by at least 30% in e.g. an animal model, such as at least 40%, at least 50%, at least 60%, or at least 80%.

In one aspect, there is provided a modified iduronate 2-sulfatase, wherein said iduronate 2-sulfatase has been prepared by sequential reaction with an alkali metal periodate and an alkali metal borohydride, thereby modifying epitopes for glycan recognition receptors of the iduronate 2-sulfatase and reducing the activity of the iduronate 2-sulfatase with respect to said glycan recognition receptors, while retaining catalytic activity of said iduronate 2-sulfatase. The iduronate 2-sulfatase is thus modified in that its epitopes, or glycan moieties, present in its natural, glycosylated form prior to modification have been essentially inactivated by said modification. The presence of epitopes for glycan recognition receptors have thus been reduced in the modified iduronate 2-sulfatase. It should be understood that the embodiments, and their advantages, disclosed in relation to the other aspects disclosed herein, such as the aspects related to modified iduronate 2-sulfatase, composition and method of preparation, are embodiments also of this aspect. In particular, the various method embodiments disclosed below provide further exemplary definition of the preparation of said modified iduronate 2-sulfatase in terms of specific reaction conditions. Similarly, the embodiments disclosed in relation to the modified iduronate 2-sulfatase and composition aspects above provide further exemplary definition of the modified iduronate 2-sulfatase.

There is, in one aspect, provided a method of preparing a modified iduronate 2-sulfatase, said method comprising:

a) reacting a glycosylated iduronate 2-sulfatase with an alkali metal periodate, and

b) reacting said iduronate 2-sulfatase with an alkali metal borohydride for a time period of no more than 2 h; thereby modifying glycan moieties of the iduronate 2-sulfatase and reducing the activity of the iduronate 2-sulfatase with respect to glycan recognition receptors, while retaining catalytic activity of said iduronate 2-sulfatase.

There is, in one aspect, provided a method of preparing a modified iduronate 2-sulfatase, said method comprising:

a) reacting a glycosylated iduronate 2-sulfatase with an alkali metal periodate, and

b) reacting said iduronate 2-sulfatase with an alkali metal borohydride for a time period of no more than 2 h; thereby modifying glycan moieties of the iduronate 2-sulfatase and reducing the activity of the iduronate 2-sulfatase with respect to glycan recognition receptors, while retaining at least 50% catalytic activity of said iduronate 2-sulfatase in vitro. Thus, the modified iduronate 2-sulfatase has a catalytic activity of at least 50% of that of unmodified iduronate 2-sulfatase in vitro.

The above method thus provides mild chemical modification of iduronate 2-sulfatase that reduces the presence of epitopes for glycan recognition receptors, said epitopes for example being represented by natural glycan moieties as described herein. This advantageously may provide a modified iduronate 2-sulfatase suitable for targeting the brain of a mammal and/or such peripheral tissues where otherwise unmodified iduronate 2-sulfatase is poorly distributed. In particular, the method may provide an iduronate 2-sulfatase with higher exposure in joints, bone, connective tissue, skeletal muscle, heart, lung and/or cartilage, when administrated by e.g. intravenous infusion. The mild method may moreover modify said epitopes without substantially altering the catalytic activity of the iduronate 2-sulfatase. In particular, catalytic activity may be retained by retaining FGly59 at the active site of iduronate 2-sulfatase. Thus, while improving distribution properties of the enzyme, the method does not eliminate catalytic activity.

Moreover, the relatively mild chemical modification may provide a modified enzyme having improved quality and stability, such as improved structural integrity. Compared to the known modification method, the modification as disclosed herein results in less protein aggregation, and thus decreased occurrence of high molecular weight forms of iduronate 2-sulfatase. Also, protein strand break is less frequent with the method as disclosed herein. Thus, less fragments of iduronate 2-sulfatase may be observed in the product resulting from the method as disclosed herein. Further advantages with the modified iduronate 2-sulfatase prepared by the mild method are as accounted for above, e.g. for the iduronate 2-sulfatase and composition aspects.

The method allows for glycan modification by periodate cleavage of carbon bonds between two adjacent hydroxyl groups of the glycan (carbohydrate) moieties. In general, periodate oxidative cleavage occurs where there are vicinal diols present. The diols have to be present in an equatorial—equatorial or axial—equatorial position. If the diols are present in a rigid axial-axial position no reaction takes place (Kristiansen et al, Car. Res (2010)). The periodate treatment will break the bond between C2 and C3 and/or C3 and C4 of the M6P moiety, thus yielding a structure that is incapable of binding to a M6P-receptor. In general, other terminal hexoses will also be processed in a similar way. Non-terminal 1-4 linked residues are cleaved between C2 and C3 only, whereas non-terminal (1-3) linked residues are resistant to cleavage. In FIG. 7 the points of possible modification are marked with an asterisk in the three general types of N-glycans; oligomannose, complex and hybrid. As further demonstrated in appended FIGS. 8-9, the method as disclosed herein provides disruption of natural glycan moieties by a limited number of bond breaks. Typically, modification by use of the prior art method give rise to more extensive disruption, as has been demonstrated in comparative experiments for the polypeptide sulfamidase (see Examples 8-9). The periodate used in step a) may disrupt the structure of the glycan moieties naturally occurring on iduronate 2-sulfatase. The remaining glycan structure of the modified iduronate 2-sulfatase may have been at least partially disrupted in that at least one periodate catalyzed cleavage, i.e. at least one single bond break, has occurred in each of the naturally occurring glycan moieties. The presently disclosed method may predominantly result in a single-type of bond breaks in sugar moieties of the glycan moieties of iduronate 2-sulfatase. A repertoire of modified glycan moieties predominantly exhibiting singe-type of bond breaks may in turn be beneficial for the distribution and activity of iduronate 2-sulfatase in the brain in a living animal after intravenous administration.

The method of preparing a modified iduronate 2-sulfatase, and the modified iduronate 2-sulfatase as described herein, are improved over prior art methods and compounds. Primarily, the novel modified iduronate 2-sulfatase may be distributed to and display catalytic activity in the mammalian brain. Examples 2 and 4 moreover provide comparisons between the known prior art method and the new methods for modification of iduronate 2-sulfatase as disclosed herein. These examples show that iduronate 2-sulfatase modified according to known methods displays at least one of amino acid residues modifications, polypeptide chain cleavages and protein aggregation. Thus, the method as disclosed herein moreover may provide a modified iduronate 2-sulfatase with improved quality and stability in terms of e.g. structural integrity

In one embodiment of the method aspect, said iduronate 2-sulfatase polypeptide comprises a polypeptide consisting of an amino acid sequence as defined in SEQ ID NO:1, or a polypeptide having sequence identity with the polypeptide defined in SEQ ID NO:1. Exemplary embodiments are further disclosed in relation to other aspects disclosed herein.

In one embodiment of the method aspect, said glycosylated iduronate 2-sulfatase contains, prior to step a), glycan moieties at eight N-glycosylation sites: asparagine (N) in position 6 (N(6)), asparagine (N) in position 90 (N(90)), N in position 119 (N(119)), N in position 221 (N(221)), N in position 255 (N(255)), N in position 300 (N(300)), N in position 488 (N(488)) and N in position 512 (N(512)) of SEQ ID NO:1.

In one embodiment of the method aspect, said alkali metal periodate oxidizes cis-glycol groups of the glycan moieties to aldehyde groups.

In one embodiment of the method aspect, said alkali metal borohydride reduces said aldehydes to alcohols.

In one embodiment of the method aspect, step a) and step b) are performed in sequence without performing an intermediate step. By performing step b) immediately after step a), or after an optional quenching step a2) as described below, any intermediate step such as to remove reactive reagents by e.g. dialysis, ultrafiltration, precipitation or buffer exchange, is omitted, and long exposure of iduronate 2-sulfatase to reactive aldehyde intermediates is thus avoided. Proceeding with step b) after step a), or optionally a2), the overall reaction duration is also advantageously reduced.

In the following paragraphs, specific embodiments for step a) is disclosed. It should be understood that unless defined otherwise specific embodiments of aspects disclosed herein can be combined.

In one embodiment, said alkali metal periodate is sodium meta-periodate.

In one embodiment, said reaction of step a) is performed for a time period of no more than 4 h, such as no more than 3 h, such as no more than 2 h, such as no more than 1 h, such as around 0.5 h. In certain embodiments, the reaction of step a) is performed for at least 0.5 h. The reaction preferably has a duration of around 3 h, 2 h, 1 h, or less than 1 h. A duration of step a) of no more than 4 hours may efficiently inactivate epitopes for glycan recognition receptors. In addition, a relatively limited duration of no more than 4 h is hypothesized to give rise to a limited degree of strand-breaks of the polypeptide chain.

In one embodiment, said periodate is used at a (final) concentration of no more than 20 mM, such as no more than 15 mM, such as around 10 mM. The periodate may be used at a concentration of 8-20 mM, preferably around 10 mM. Alternatively, the periodate is used at a concentration of less than 20 mM, such as between 10 and 19 mM. Lower concentration of alkali metal periodate, such as sodium meta-periodate, may reduce the degree of strand-breaks of the polypeptide chain, as well as associated oxidation on amino acids side-chains, such as oxidation of the methionines.

In one embodiment, said reaction of step a) is performed at ambient temperature, and preferably at a temperature of between 0 and 22° C. In a preferred embodiment, the reaction of said step a) is performed at a temperature of 0-8° C., such as at a temperature of 0-4° C. In a preferred embodiment, the reaction of step a) is performed at a temperature of around 8° C., at a temperature of around 4° C. or at a temperature of around 0° C.

In one embodiment, said reaction of step a) is performed at a pH of 3 to 7. This pH should be understood as the pH at the initiation of the reaction. In particular embodiments, the pH used in step a) is 3-6, such as 4-5. In specific embodiments, the pH used in step a) is around 6, around 5, or around 4. By lowering the pH of step a), the concentration of periodate or the reaction time of step a) may be reduced.

In one embodiment, said periodate is sodium meta-periodate and is used at a (final) concentration of no more than 20 mM, such as no more than 15 mM, such as around 10 mM. In one embodiment, said sodium meta-periodate is used at a concentration of 8-20 mM. In preferred embodiments, sodium meta-periodate is used at a concentration of around 10 mM.

In one embodiment, said periodate is sodium meta-periodate and is used at a (final) concentration of no more than 20 mM, such as no more than 15 mM, such as around 10 mM, and said reaction of step a) is performed for a time period of no more than 4 h, such as no more than 3 h, such as no more than 2 h, such as no more than 1 h, such as around 0.5 h. A concentration of 20 mM periodate and a reaction duration of no more than 4 h may advantageously result in less strand-break and oxidation. Decreasing the periodate concentration further while maintaining the relatively short reaction duration may positively affect strand-break and oxidation further. Moreover, decreasing the periodate concentration further while maintaining the relatively short reaction duration may positively affect the covalently linking of iduronate 2-sulfatase monomeric subunits (i.e. decrease the occurrence of covalently linked monomers).

In one embodiment, said periodate is sodium meta-periodate and is used at a (final) concentration of no more than 20 mM, such as no more than 15 mM, such as around 10 mM, and said reaction of step a) is performed for a time period of no more than 4 h, such as no more than 3 h, such as no more than 2 h, such as no more than 1 h, such as around 0.5 h at a temperature of between 0 and 22° C., such as around 8° C., such as around 0° C.

In one embodiment, said periodate is used at a concentration of no more than 20 mM, such as no more than 15 mM, such as around 10 mM, and said reaction of step a) is performed for a time period of no more than 4 h, such as no more than 3 h, such as no more than 2 h, such as no more than 1 h, such as around 0.5 h, at a temperature of between 0 and 22° C., such as a temperature of 0-8° C., such as a temperature of 0-4° C., such as around 8° C., such as around 0° C.

In one embodiment, said periodate is sodium meta-periodate and said reaction of step a) is performed for a time period of no more than 4 h, such as no more than 3 h, such as no more than 2 h, such as no more than 1 h, such as around 0.5 h at a temperature of between 0 and 22° C., such as a temperature of 0-8° C., such as a temperature of 0-4° C., such as around 8° C., such as around 0° C.

In one embodiment, said periodate is sodium meta-periodate which is used at a concentration of no more than 20 mM, such as no more than 15 mM, such as around 10 mM, and said reaction of step a) is performed at a temperature of between 0 and 22° C., such as a temperature of 0-8° C., such as a temperature of 0-4° C., such as around 8° C., such as around 0° C.

In one embodiment, said periodate is sodium meta-periodate which is used at a concentration around 10 mM, and said reaction of step a) is performed at a temperature of around 8° C. and for a time period of no more than 2 h.

In one embodiment, said periodate is sodium meta-periodate which is used at a concentration of around 10 mM, and said reaction of step a) is performed at a temperature of 0-8° C. and for a time period of no more than 3 h.

In the following paragraphs, specific embodiments of step b) are disclosed. It should be understood that unless defined otherwise, specific embodiments can be combined, in particular specific embodiments of step a) and step b).

In one embodiment, said borohydride is optionally used at a concentration of between 10 and 80 mM, such as at a concentration of between 10 and 80 mM.

In one embodiment, said alkali metal borohydride is sodium borohydride.

In some instances, the conditions used for step b) have been found to partly depend on the conditions used for step a). While the amount of borohydride used in step b) is preferably kept as low as possible, the molar ratio of borohydride to periodate is in such instances 0.5-4 to 1. Thus, borohydride may in step b) be used in a molar excess of 4 times the amount of periodate used in step a). In one embodiment, said borohydride is used at a (final) molar concentration of no more than 4 times the (final) concentration of said periodate. For example, borohydride may be used at a concentration of no more than 3 times the concentration of said periodate, such as no more than 2.5 times the concentration of said periodate, such as no more than 2 times the concentration of said periodate, such as no more than 1.5 times the concentration of said periodate, such as at a concentration roughly corresponding to the concentration of said periodate. However, in particular embodiments borohydride is used at a concentration corresponding to half of the periodate concentration, or 0.5 times the periodate concentration. Thus, when periodate is used at a concentration of around 20 mM, borohydride might be used at a concentration of no more than 80 mM, or even at a concentration between 10 and 80 mM, such as at a concentration of between 10 and 50 mM. If periodate is used at a concentration of between 10 and 20 mM, borohydride might be used at a concentration of between 5 and 80 mM, such as for example 50 mM. Similarly, if periodate is used at a concentration of around 10 mM, borohydride might be used at a concentration of no more than 40 mM, such as for example no more than 25 mM. Moreover, in such an embodiment, borohydride may preferably be used at a concentration of between 12 mM and 50 mM. The concentration of borohydride may influence the degree of preservation of a catalytic amino acid residue at the active site of iduronate 2-sulfatase, hence a relatively lower concentration of borohydride may provide a modified iduronate 2-sulfatase having retained catalytic activity.

In one embodiment, said reaction of step b) is performed for a time period of no more than 1.5 h, such as no more than 1 h, such as no more than 0.75 h, such as around 0.5 h. The reaction duration is preferably around 1 h, or less than 1 h. In some instances, the reaction of step b) has a duration of approximately 0.25 h. In further embodiments, the reaction of step b) may be performed for a time period of from 0.25 h to 2 h. As accounted for above, the duration of the reduction step may affect the catalytic activity of the iduronate 2-sulfatase. A relatively short reaction duration may thus provide a modified iduronate 2-sulfatase comprising FGly59 rather than Ser59. A shorter reaction duration may moreover favorably influence the overall structural integrity of the enzyme. In particular, protein aggregation resulting in high molecular weight forms of iduronate 2-sulfatase as well as strand-break occurrence may at least partly be related to reaction time.

In one embodiment, said reaction of step b) is performed at a temperature of between 0 and 8° C. Reaction temperature for step b) may at least partly affect catalytic activity of the reaction product. Thus, it may be advantageous to perform step b) at a temperature of below 8° C. The temperature is preferably around 0° C.

In one embodiment, said alkali metal borohydride is sodium borohydride which is used at a concentration of 0.5-4 times the concentration of said periodate, such as at a concentration of no more than 2.5 times the concentration of said periodate.

In one embodiment, said alkali metal borohydride is sodium borohydride which is used at a concentration of 0.5-4 times the concentration of said periodate, such as at a concentration of no more than 2.5 times the concentration of said periodate, and said reaction of step b) is performed for a time period of no more than 1 h, such as around 0.5 h.

In one embodiment, said alkali metal borohydride is sodium borohydride which is used at a concentration of 0.5-4 times the concentration of said periodate, such as at a concentration of no more than 2.5 times the concentration of said periodate, and said reaction of step b) is performed for a time period of no more than 1 h, such as around 0.5 h, at a temperature of between 0 and 8° C.

In one embodiment, said alkali metal borohydride is used at a concentration of 0.5-4 times the concentration of said periodate, such as at a concentration of no more than 2.5 times the concentration of said periodate, and said reaction of step b) is performed for a time period of no more than 1 h, such as around 0.5 h, at a temperature of between 0 and 8° C.

In one embodiment, said alkali metal borohydride is sodium borohydride, and said reaction of step b) is performed for a time period of no more than 1 h, such as around 0.5 h, at a temperature of between 0 and 8° C.

In one embodiment, said alkali metal borohydride is sodium borohydride which is used at a concentration of 0.5-4 times the concentration of said periodate, such as at a concentration of no more than 2.5 times the concentration of said periodate, and said reaction of step b) is performed at a temperature of between 0 and 8° C.

In one embodiment, said alkali metal borohydride is sodium borohydride which is used at a concentration of 0.5-4 times the concentration of said periodate, such as at a concentration of 2.5 times the concentration of said periodate, and said reaction of step b) is performed at a temperature of around 0° C. for a time period of around 0.5 h.

In one embodiment, said periodate is sodium meta-periodate and said alkali metal borohydride is sodium borohydride.

In one embodiment, each of step a) and step b) is individually performed for a time period of no more than 2 h, such as no more than 1 h, such as around 1 h or around 0.5 h. Optionally, said borohydride is used at a concentration of 0.5-4 times the concentration of said periodate, preferably 0.5-2.5 times the concentration of said periodate. In certain embodiments, said borohydride is used at a concentration of 0.5 times the concentration of the periodate, or at a concentration of 2.5 times the concentration of said periodate.

In one embodiment, step a) is performed for a time period of no more than 3 h and step b) is performed for no more than 1 h. Optionally, said borohydride is used at a concentration of no more than 4 times the concentration of said periodate, preferably no more than 2.5 times the concentration of said periodate.

The person skilled in the art is aware of ways to control the reaction duration of a chemical reaction, such as the reaction duration of each of step a) and b). Thus, in one embodiment, said method aspect further comprises a2) quenching of the reaction resulting from step a). Said quenching for example has a duration of less than 30 minutes, such as less than 15 minutes. In some instances, said quenching is performed immediately after step a). Quenching may for example be performed by addition of ethylene glycol or another diol, such as for example cis-cyclo-heptane-1,2-diol. Preferably, step b) follows immediately after the quenching. This may minimize the period of exposure for iduronate 2-sulfatase to reactive aldehyde groups. Reactive aldehydes can promote inactivation and aggregation of the protein.

In one embodiment, said method further comprises b2) quenching of the reaction resulting from step b). This quenching may for example be conducted by addition of a molecule that contains a ketone or aldehyde group, such as cyclohexanone or acetone, said molecule preferably being soluble in water, or by lowering the pH below 6 of the reaction mixture by addition of acetic acid or another acid. In some instances, said quenching is performed by addition of acetone. An optional quenching step allows for a precise control of reaction duration for step b). Controlling reaction duration in this way may further provide reproducibility of the process in terms of FGly59 content.

In some instances, chemical modification may affect the activity of the enzyme. In order to minimize such negative effects of chemical modification, the active site of said iduronate 2-sulfatase can be made inaccessible to oxidative and/or reductive reactions during at least one of steps a) and b). Thus, in one embodiment, at least one of steps a) and b) of the method is/are performed in the presence of a protective ligand. In particular, step a) may be performed in presence of a protective ligand. A ligand, such as a substrate to iduronate 2-sulfatase, e.g. 4-methylumbeliferone iduronide-sulfate or heparin sulfate, or an inhibitor, such as a sulfate, may serve to protect the active site of iduronate 2-sulfatase during the step(s) of oxidation and/or reduction, and optionally the quenching step(s). In another embodiment, steps a) and b) of the method are performed while iduronate 2-sulfatase is immobilized on a resin. Thus, iduronate 2-sulfatase may initially be immobilized on a resin or medium. Then the reactions of steps a) and b), and optionally a2) and b2), may be conducted while iduronate 2-sulfatase is immobilized onto the resin or medium. Suitable resins or mediums are known to the skilled person. For example, anion exchange media or affinity media may be used.

In one embodiment, steps a) and b) of the method are performed in a continuous process. The term “continuous process” as used herein should be understood as a process that is continuously operated and wherein reagents are continuously fed to the process unit. In particular, steps a), a2), b), and b2) may be performed in a continuous process. By adding the reagents, such as the alkali metal periodate and the alkali metal borohydride, to a stream of iduronate 2-sulfatase, the reaction can be carried out in a continuous mode. A continuous process can for example be carried out in a multi-pump HPLC system.

The method as disclosed herein thus provides a modified iduronate 2-sulfatase having improved properties. It is expected that the conditions for chemical modification of iduronate 2-sulfatase provides minimal negative impact on structural integrity of the iduronate 2-sulfatase polypeptide chain, and simultaneously results in substantial absence of natural glycan structures suggesting a nearly complete modification of glycans at all eight natural glycosylated sites while retaining catalytic activity. Exemplary embodiments of the method are depicted in FIGS. 1B, 1C and 1D.

In a related aspect, there is provided a method of producing iduronate 2-sulfatase, said method comprising:

expressing said iduronate 2-sulfatase in mammalian, plant or yeast cells, thereby providing a glycosylated iduronate 2-sulfatase, and

modifying epitopes for glycan recognition receptors on said glycosylated iduronate 2-sulfatase, thereby reducing the activity of the iduronate 2-sulfatase with respect to said glycan recognition receptors. One example of a cell line is a CHO cell line.

In one embodiment, said modifying is conducted by sequential reaction with an alkali metal periodate and an alkali metal borohydride. Other embodiments of said method are disclosed above.

In one aspect, there is provided a modified iduronate 2-sulfatase obtainable by any one of the methods disclosed herein.

In one aspect, there is provided a modified iduronate 2-sulfatase obtainable by any one of the methods disclosed herein for use in therapy.

In one aspect, there is provided a modified iduronate 2-sulfatase obtainable by any one of the methods disclosed herein for use in treatment of lysosomal storage disease, in particular mucopolysaccharidosis II (MPS-II; Hunter syndrome).

In one aspect, use of a modified iduronate 2-sulfatase in the manufacture of a medicament is provided, for crossing the blood brain barrier to treat a lysosomal storage disease, such as mucopolysaccharidosis II (MPS-II; Hunter syndrome), in a mammalian brain, said modification comprises having glycan moieties chemically modified by sequential treatment of the enzyme with an alkali metal periodate and an alkali metal borohydride, thereby reducing the activity of the iduronate 2-sulfatase with respect to glycan recognition receptors, such as mannose and mannose-6-phosphate cellular delivery systems, while retaining catalytic activity of said iduronate 2-sulfatase.

In one aspect, use of a modified iduronate 2-sulfatase in the manufacture of a medicament is provided, for enhanced distribution to affected visceral organs in a mammal to treat a lysosomal storage disease, such as mucopolysaccharidosis II (MPS-II; Hunter syndrome), in said affected visceral organs, said modification comprises having glycan moieties chemically modified by sequential treatment of the enzyme with an alkali metal periodate and an alkali metal borohydride, thereby reducing the activity of the iduronate 2-sulfatase with respect to glycan recognition receptors, such as mannose and mannose-6-phosphate cellular delivery systems, while retaining catalytic activity of said iduronate 2-sulfatase.

In one aspect there is provided a method of treating a mammal afflicted with a lysosomal storage disease, such as mucopolysaccharidosis II (MPS-II; Hunter syndrome), comprising administering to the mammal a therapeutically effective amount of a modified iduronate 2-sulfatase, said modified iduronate 2-sulfatase being selected from:

a) a modified iduronate 2-sulfatase as described in aspects and embodiments herein, and

b) an iduronate 2-sulfatase composition as described in aspects and embodiments herein.

In one embodiment thereof, said treatment results in clearance of about at least 50% lysosomal storage from the brain of a mammal after administration of 5 doses of modified iduronate 2-sulfatase over a time period of 35 days.

The invention will be further illustrated by the following non-limiting examples.

### BRIEF DESCRIPTION OF THE DRAWINGS

FIG. 1 is a picture outlining the differences between the methods for chemical modification developed by the inventors, disclosed in Example 3, and the known method, disclosed in WO 2008/109677.

FIG. 2A shows a SDS-PAGE gel of iduronate 2-sulfatase (lane 2) and iduronate 2-sulfatase modified according to the known method (lane 3). Two additional protein bands, denoted A and B, generated by the glycan modification procedure were identified in lane 3.

FIG. 2B shows a SDS-PAGE gel of iduronate 2-sulfatase (lane 2), iduronate 2-sulfatase modified according to the known method (lane 3) as well as iduronate 2-sulfatase modified according to new method 1, 2, 3, 4 as disclosed herein (lanes 4, 5, 6, 7, respectively).

FIG. 3 is a diagram showing the relative amounts of different naturally occurring glycans at asparagine in position 90 (N(90)) of a peptide fragment of iduronate 2-sulfatase (black bars), iduronate 2-sulfatase modified according to the known method (grey bars) as well as iduronate 2-sulfatase modified according to new method 1 (checkered bars). The glycans correspond to the following; GOF: Asialo-, agalacto-, fucosylated biantennary oligosaccharide (Oxford notation name: FA2); G1F: Monogalactosylated, fucosylated biantennary oligosaccharide (Oxford notation name: FA2[3]G1 or FA2[6]G1); G2F: Asialo-, fucosylated biantennary oligosaccharide (Oxford notation name: FA2G2); A1F: Monosialo-, fucosylated biantennary oligosaccharide (Oxford notation name: FA2G2S1); A2F: Disialo-, fucosylated biantennary oligosaccharide (Oxford notation name: FA2G2S2).

FIG. 4 is a diagram showing the activity of iduronate 2-sulfatase as well as iduronate 2-sulfatase modified according to new method 3 and 4.

FIG. 5 is a diagram visualizing the receptor mediated endocytosis in human primary fibroblast cells of unmodified recombinant iduronate 2-sulfatase (black squares) and iduronate 2-sulfatase modified according to new method 1 as described herein (black circles).

FIG. 6A shows the time dependence of serum concentrations of iduronate 2-sulfatase and iduronate 2-sulfatase chemically modified according to new method 2 in mice after i.v. administration at a dose of 1 mg/kg.

FIG. 6B shows the time dependence of serum concentrations of iduronate 2-sulfatase and iduronate 2-sulfatase chemically modified according to new method 3 in mice after i.v. administration at a dose of 3 mg/kg.

FIG. 7 is a schematic drawing of the three archetypal N-glycan structures generally present in proteins of mammalian origin and the typical N-glycan present in yeast proteins. The left glycan represents the oligomannose type, the second from the left the complex type, and the second from right, the hybrid type and the one on the far right is the polymannose type of yeast proteins. In the Figure the following compounds are depicted: black filled diamonds correspond to N-acetylneuraminic acid; black filled circles correspond to mannose; squares correspond to N-acetylglucosamine; black filled triangle corresponds to fucose; circle corresponds to galactose. Sugar moieties marked with an asterisk can be modified by the periodate/borohydride treatment disclosed herein.

FIG. 8A is a schematic drawing illustrating predicted bond breaks on mannose after chemical modification.

FIG. 8B is a schematic drawing illustrating a model of a Man-6 glycan. The sugar moieties susceptible to bond breaks upon oxidation with periodate are indicated. Grey circles correspond to mannose, black squares correspond to N-acetylglucosamine, T13 corresponds to the tryptic peptide NITR including the N-glycosylation site N(131) of SEQ ID NO:2 of the related enzyme sulfamidase.

FIG. 9A is a diagram visualizing the extent of bond breaking of the tryptic peptide T13+Man-6 glycan after chemical modification of sulfamidase, a related lysosomal enzyme, according to the previously known method (black bar), new method 1 (black dots), new method 2 (white), and new method 3 (cross-checkered).

FIG. 9B is a diagram visualizing the relative abundance of single bond breaks in the tryptic peptide T13+Man-6 glycan after chemical modification of sulfamidase according to the previously known method (black bar), new method 1 (black dots), new method 2 (white), and new method 3 (cross-checkered).

FIG. 10 is a table listing amino acid sequences of human iduronate 2-sulfatase, (SEQ ID NO:1) and human sulfamidase (SEQ ID NO:2; related to Examples 8 and 9).

### EXAMPLES

The Examples which follow disclose the development of a modified iduronate 2-sulfatase polypeptide according to the present disclosure.

Unless stated otherwise, the recombinant iduronate 2-sulfatase used in the Examples below was the medicinal product Elaprase®. Elaprase® was purchased from a pharmacy (Apoteket farmaci, Sweden), stored according to the manufacturer's specifications and treated under sterile conditions.

### Example 1

### Chemical Modification of Iduronate 2-Sulfatase According to Previously Known Method

### Material and Methods

Prior to chemical modification iduronate 2-sulfatase was diluted to 0.58 mg/ml in Elaprase® drug product buffer.

### Chemical Modification According to WO 2008/109677:

In order to modify glycan moieties, iduronate 2-sulfatase (SEQ ID NO:1), was initially incubated with 20 mM sodium meta-periodate at 0° C. for 6.5 h in 20 mM sodium phosphate, 137 mM NaCl (pH 6.0). Glycan oxidation was quenched by addition of ethylene glycol to a final concentration of 192 mM. Quenching was allowed to proceed for 15 min at 0° C. before performing dialysis against 20 mM sodium phosphate, 137 mM NaCl (pH 6.0) over night at 4° C. Following dialysis, reduction was performed by addition of sodium borohydride to the reaction mixture at a final concentration of 100 mM. The reduction reaction was allowed to proceed over night. Finally, the enzyme preparation was dialyzed against 20 mM sodium phosphate, 137 mM NaCl (pH 6.0). All incubations were performed in the dark.

### Example 2

### Analyses of Iduronate 2-Sulfatase Modified According to Known Method

### Material and Methods

The iduronate 2-sulfatase modified according to known method as described in Example 1 was subjected to the following analyses.

### SDS-PAGE Analysis:

5 μg of iduronate 2-sulfatase and modified iduronate 2-sulfatase was loaded into each well on a NuPAGE 4-12% Bis-Tris gel. Seeblue 2 plus marker was used and the gel was colored with Instant Blue (C.B.S Scientific).

### Enzymatic Activity:

Catalytic activity of iduronate 2-sulfatase was assessed by incubating preparations of iduronate 2-sulfatase with the substrate 4-Methylumbeliferone iduronide-sulfate. The concentration of substrate in the reaction mixture was 50 μM and the assay buffer was 50 mM sodium acetate, 0.005% Tween 20, 0.1% BSA, 0.025% Anapoe X-100, 1.5 mM sodium azide, pH 5. After the incubation, further desulphation was inhibited by addition of a stop buffer containing 0.4 M sodium phosphate, 0.2 M citrate pH 4.5. A second 24 hour incubation with iduronate 2-sulfatase (assay concentration 0.83 μg/mL) was performed to hydrolyze the product (4-methylumbeliferone iduronide) and release 4-Methylumbeliferone, which was monitored by fluorescence at 460 nm after quenching the reaction with 0.5 M sodium carbonate, 0.025% Triton X-100, pH 10.7.

### Glycan Analysis by LC/MS of Tryptic Fragments:

The glycosylation pattern was determined for the different iduronate-2-sulfatase batches produced. Prior to glycopeptide analysis, iduronate-2-sulfatase (ca 20 μg) was reduced, alkylated and digested with trypsin. Reduction of the protein was done by incubation in 5 μl DTT 10 mM in 50 mM NH4HCO3 at 60° C. for 1 h. Subsequent alkylation with 5 μl iodoacetamide 55 mM in 50 mM NH₄HCO₃ was performed at room temperature (RT) and in darkness for 45 min. Lastly, the tryptic digestion was performed by addition of 30 μl of 50 mM NH₄HCO₃, 5 mM CaCl₂, pH 8, and 0.2 μg/μl trypsin in 50 mM acetic acid (protease: protein ratio 1:20 (w/w)). Digestion was allowed to take place over night at 37° C.

Seven peptide fragments of the trypsin digested iduronate-2-sulfatase contained potential N-glycosylation sites, N(x), where x refers the position of the asparagine in the iduronate-2-sulfatase amino acid (aa) sequence as defined in SEQ ID NO:1, were:

N(6) peptide, aa 1-23, 2500.30 Da
N(90) peptide, aa 86-99, 1607.81 Da
N(119) peptide, aa 111-139, 3301.47 Da
N(221) peptide, aa 216-246, 3504.76 Da
N(255) peptide, aa 249-269, 2356.21 Da
N(300) peptide, aa 289-322, 3678.83 Da
N(488) and N(512) peptide, aa 474-525, 5980.70 Da
The molecular mass of each peptide fragment is given.

For the investigation of possible glycosylation variants, the N(90) tryptic peptide fragment was selected for further glycopeptide analysis. The analysis was performed by liquid chromatography followed by mass spectrometry (LC-MS) on an Agilent 1200 HPLC system coupled to an Agilent 6510 Quadrupole time-of-flight mass spectrometer (Q-TOF-MS, Agilent Technologies). Both systems were controlled by MassHunter Workstation. LC separation was performed by the use of a Waters XSELECT CSH 130 C18 column (150×2.1 mm), the column temperature was set to 40° C. Mobile phase A consisted of 5% acetonitrile, 0.1% propionic acid, and 0.02% TFA, and mobile phase B consisted of 95% acetonitrile, 0.1% propionic acid, and 0.02% TFA. A gradient of from 0% to 10% B for 10 minutes, then from 10% to 70% B for another 25 min was used at a flow rate of 0.2 mL/min. The injection volume was 10 μl. The Q-TOF MS was operated in positive-electrospray ion mode. During the course of data acquisition, the fragmentor voltage, skimmer voltage, and octopole RF were set to 90, 65, and 650 V, respectively. Mass range was between 300 and 2800 m/z.

### Results

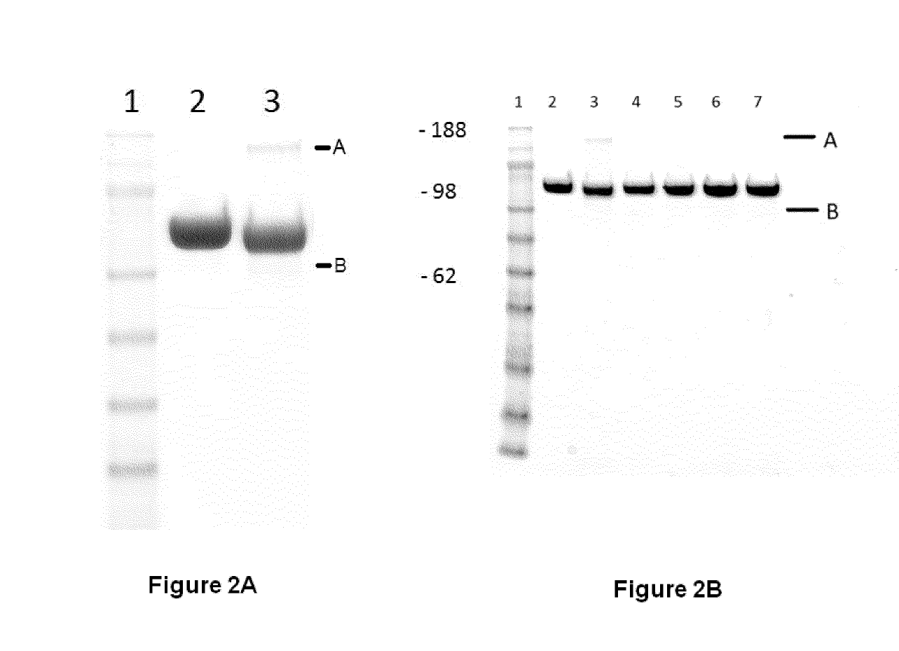

As apparent by SDS-PAGE analysis, two major peptides of sizes distinct from that of monomeric iduronate 2-sulfatase were formed as a result of the chemical modification (FIG. 2A, lane 3). The first peptide denoted A is roughly twice the size of monomeric iduronate 2-sulfatase, and most probably represents a covalently linked dimer, whereas the second peptide denoted B is roughly ˜5 kDa smaller than iduronate 2-sulfatase and represents a peptide cleavage product. The main peptide band, representing the monomer, is smaller for iduronate 2-sulfatase modified using the known method as compared to the unmodified iduronate 2-sulfatase, indicative of loss of molecular weight by the chemical modification procedure (FIG. 2A, lane 3 versus lane 2). The molecular weight of iduronate 2-sulfatase is somewhat reduced after modification due to the bond breaking within the glycan moieties.

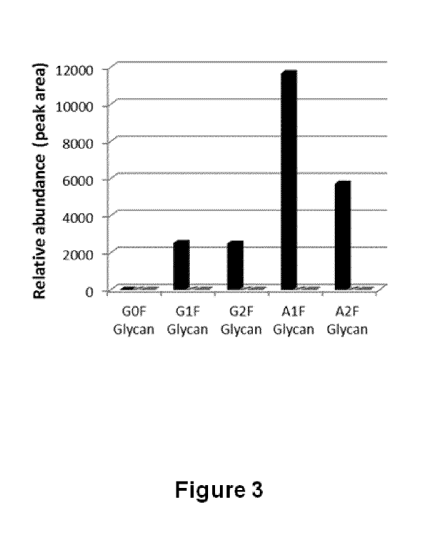

Glycan analysis was performed on the selected N(90) tryptic peptide fragment both prior and after chemical modification. Prior to chemical modification sialylated and fucosylated complex oligosaccharides were found on this asparagine. After the chemical modification no naturally occurring glycan structures were present at this position (FIG. 3).

The activity of iduronate 2-sulfatase modified according to the known method was below 50% of that of unmodified iduronate 2-sulfatase (results not shown).

### Example 3

### New Methods for Chemical Modification of Iduronate 2-Sulfatase

### Material and Methods

Prior to chemical modification iduronate 2-sulfatase was diluted to 0.58 mg/ml in Elaprase® drug product buffer.

### Chemical Modification According to New Method 1:

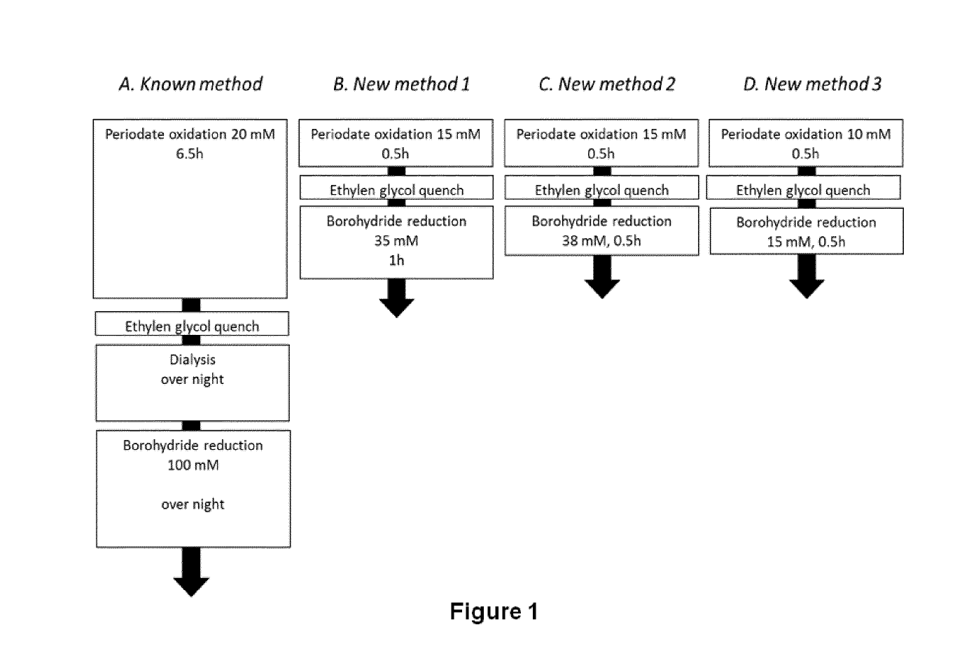

Iduronate 2-sulfatase was initially incubated at 15 mM sodium meta-periodate at 0° C. for 1 h in 20 mM sodium phosphate, 137 mM NaCl (pH 6.0). Glycan oxidation was quenched by addition of ethylene glycol to a final concentration of 192 mM. Quenching was allowed to proceed for 15 min at 0° C. Thereafter sodium borohydride was added to the reaction mixture to a final concentration of 35 mM and was allowed to proceed for 1.5 h at 4° C. Finally, the enzyme preparation was ultrafiltrated against 20 mM sodium phosphate, 137 mM NaCl (pH 6.0). All incubations were performed in the dark. The new method 1 for chemical modification is depicted in FIG. 1B.

### Chemical Modification According to New Method 2:

Iduronate 2-sulfatase was initially incubated at 15 mM sodium meta-periodate at 0° C. for 0.5 h in 20 mM sodium phosphate, 137 mM NaCl (pH 6.0). Glycan oxidation was quenched by addition of ethylene glycol to a final concentration of 96 mM. Quenching was allowed to proceed for 15 min at 0° C. Thereafter sodium borohydride was added to the reaction mixture to a final concentration of 38 mM and the resulting mixture was held at 0° C. for 0.5 h. Finally, the enzyme preparation was ultrafiltrated against 20 mM sodium phosphate, 137 mM NaCl (pH 6.0). All incubations were performed in the dark. The new method 2 for chemical modification is depicted in FIG. 1C.

### Chemical Modification According to New Method 3:

Iduronate 2-sulfatase was initially incubated at 10 mM sodium meta-periodate at 0° C. for 0.5 h in 20 mM sodium phosphate, 137 mM NaCl (pH 6.0). Glycan oxidation was quenched by addition of ethylene glycol to a final concentration of 96 mM. Quenching was allowed to proceed for 15 min at 0° C. Thereafter sodium borohydride was added to the reaction mixture to a final concentration of 15 mM and the resulting mixture was held at 0° C. for 0.5 h. Finally, the enzyme preparation was ultrafiltrated against 20 mM sodium phosphate, 137 mM NaCl (pH 6.0). All incubations were performed in the dark. The new method 3 for chemical modification is depicted in FIG. 1D.

### Chemical Modification According to New Method 4:

Reaction conditions were as described for new method 2, with the single exception that periodate oxidation was performed in the presence of 0.5 mg/mL heparin.

### Results

As already accounted for elsewhere herein, sodium meta-periodate is an oxidant that converts cis-glycol groups of carbohydrates to aldehyde groups, whereas borohydride is a reducing agent that reduces the aldehydes to more inert alcohols. The carbohydrate structure is thus irreversibly destroyed.

In order to provide an improved method for chemical modification of glycans, in particular a procedure that provides a modified iduronate 2-sulfatase with improved properties, different reaction conditions were evaluated. It could be concluded that both oxidation by sodium meta-periodate and reduction by sodium borohydride introduced polypeptide modifications and aggregation; properties that negatively impact on catalytic activity and immunogenic propensity.

Conditions were discovered for an improved chemical modification procedure. Surprisingly, these conditions facilitated that the reduction step could be performed immediately after the ethylene glycol quenching step, omitting buffer change and long exposure of iduronate 2-sulfatase to reactive aldehyde intermediates. The new chemical modification procedures are depicted in FIG. 1B, FIG. 1C and FIG. 1D.

### Example 4

### Analyses of Iduronate 2-Sulfatase Modified According to New Methods

### Material and Methods

The iduronate 2-sulfatase modified according to the new methods of Example 3 were subjected to the following analyses.

### SDS-PAGE Analysis:

2 μg of iduronate 2-sulfatase modified in accordance with the known method (Example 1) as well as with the new method 1, 2, 3 and 4 (Example 3) were loaded into separate individual wells in accordance with the description in Example 2.

### Glycan Analysis by LC/MS of Tryptic Fragments:

The glycan analysis was performed as described in Example 2.

### Enzymatic Activity:

Activity was determined according to the procedure described in Example 2.

### Results

### SDS-PAGE Analysis:

The new chemical modification methods 1, 3-4 (FIG. 2B, lane 4, 6 and 7) resulted in single peptides of sizes identical to that of unmodified full length iduronate 2-sulfatase. New method 2 (FIG. 2B, lane 5) however also resulted in a peptide corresponding to the band denoted A in FIG. 2B. However, compared to the iduronate 2-sulfatase modified according to the known method, new method 2 gave rise to less unwanted covalent dimerization. Thus, the decrease in monomer size, which was apparent for the iduronate 2-sulfatase modified with the known method, was not observed for any of the new methods 1-4. In addition, strand-breaks in the iduronate 2-sulfatase polypeptide prepared by the new methods could not be observed or were very limited compared to strand-break occurrence in the iduronate 2-sulfatase prepared according to Example 1. Importantly, the use of a ligand protecting the active site (heparin in new method 4) was compatible with the procedure and resulted in modified iduronate 2-sulfatase that by SDS-PAGE analysis was indistinguishable from that where the ligand was omitted (new method 3).

In conclusion, process related impurities, limiting the quality and safety of a medicament produced by the modification methods, are significantly reduced by the new methods as compared to the previously known methods.

### Glycan Analysis by LC/MS of Tryptic Fragments:

Glycan analysis of the selected N(90) tryptic peptide fragment showed that no naturally occurring glycan structures were present at this position after chemical modification (FIG. 3).

### Enzymatic Activity:

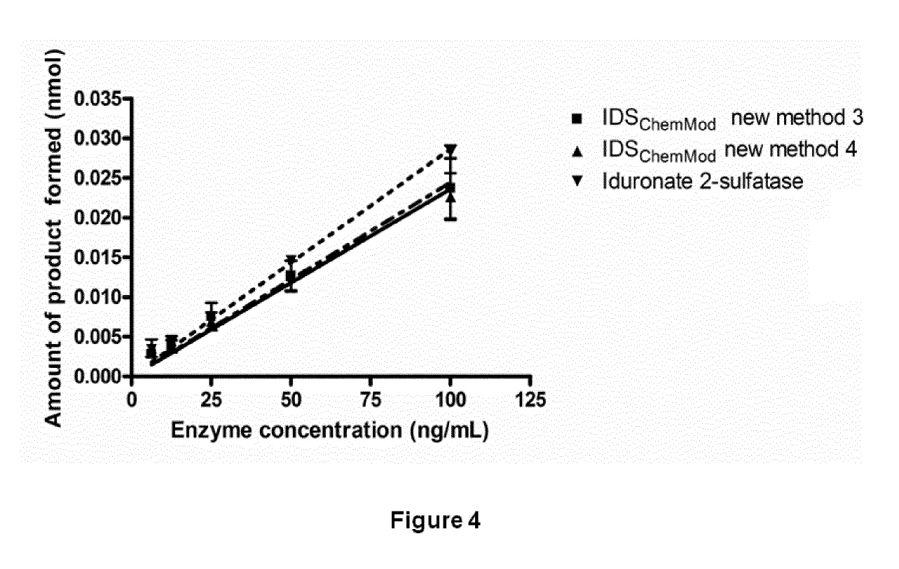

Iduronate 2-sulfatase prepared according to new method 1, 2, 3 and 4 showed an activity that was comparable to that of unmodified iduronate 2-sulfatase (FIG. 4).

### Example 5

### Receptor Mediated Endocytosis In Vitro

### Material and Methods

Endocytosis of Iduronate 2-sulfatase and Iduronate 2-sulfatase modified according to the new method 1 was evaluated in human primary fibroblasts expressing M6P receptors. The fibroblast cells were incubated for 24 h in DMEM medium supplemented with iduronate 2-sulfatase (2, 0.5 and 0.12 μg/mL), Iduronate 2-sulfatase modified according to the new method 1 (4, 1 and 0.25 μg/mL) or PBS. The cells were washed twice in DMEM and once in 0.9% NaCl prior to cell lysis using 100 μL 1% Triton X100. Lysate iduronate 2-sulfatase protein content was determined using the electrochemiluminescence immunoassay described in Example 6.

### Results

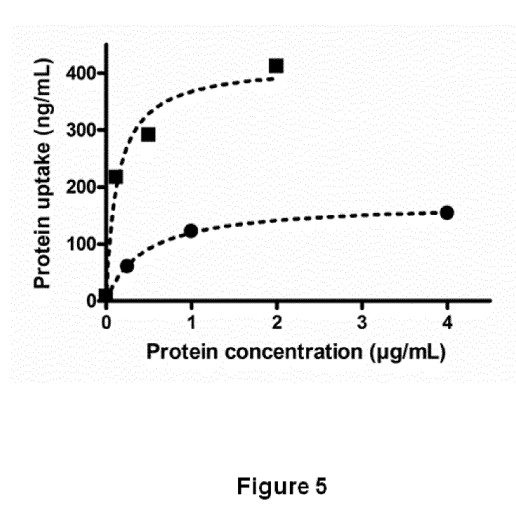

Iduronate 2-sulfatase could be detected in cell homogenate for both preparations evaluated in the endocytosis assay. Modified iduronate 2-sulfatase prepared by new method 1 had a protein concentration in cell homogenate below 25% of that obtained with unmodified recombinant iduronate 2-sulfatase (FIG. 5). The protein concentration retained in cells first loaded with and then grown in the absence of iduronate 2-sulfatase for 2 days were comparable for all preparations showing that chemical modification do not negatively impact on lysosomal stability.

It can therefore be concluded that chemical modification render iduronate 2-sulfatase less prone to cellular uptake which is a consequence of removal of epitopes for glycan recognition receptors such as M6PR. On a macroscopic level, this loss of molecular interactions translates into a reduced clearance from plasma when administrated intravenously. The reduced clearance of the protein could allow for less frequent dosing for the patients.

### Example 6

### In Vivo Serum Clearance of Modified Iduronate 2-Sulfatase Produced by New Method 2 & 3

### Material and Methods

Serum clearance (CL) of unmodified and modified recombinant iduronate 2-sulfatase modified according to the new method 2 and 3 of Example 3 was investigated in mice (C57BL/6J). The mice were given an intravenous single dose administration in the tail vein. Iduronate 2-sulfatase modified according to the new method 2 was studied together with unmodified iduronate 2-sulfatase at a dose of 1 mg/kg. Both enzymes were formulated at 0.2 mg/mL and administered at 5 mL/kg. Iduronate 2-sulfatase modified according to the new method 3 was studied together with unmodified iduronate 2-sulfatase at a dose of 3 mg/kg. Both enzymes were formulated at 0.6 mg/mL and administered at 5 mL/kg. Blood samples were taken at different time points up to 24 h post dose (3 mice per time point). The serum levels of iduronate 2-sulfatase and modified iduronate 2-sulfatase were analyzed by ECL. Serum clearance was calculated using WinNonlin software version 6.3 (Non-compartmental analysis, Phoenix, Pharsight Corp., USA).

### Quantification of Iduronate 2-Sulfatase and Modified Iduronate 2-Sulfatase by Electrochemiluminescence (ECL) Immunoassay:

Iduronate 2-sulfatase and modified iduronate 2-sulfatase in serum PK samples were determined by ECL immunoassay using the Meso Scale Discovery (MSD) platform. The wells of a 96 well streptavidin gold plate (#L15SA-1, MesoScaleDiscovery (MSD)) were blocked with 1% Fish Gelatin in Phosphate buffer saline (PBS), washed with wash buffer (PBS+0.05% Tween-20) and incubated with a biotinylated, affinity purified goat-a-human Iduronate 2-sulfatase polyclonal antibody (BAF2449, R&D) after washing different dilutions of standard and PK samples in sample diluent (1% Fish Gelatin in PBS+0.05% Tween 20+1% C57BL6 serum pool) were incubated in the plate at 700 rpm shake and RT for 2 h. The plate was washed and a iduronate 2-sulfatase specific Rutenium (SULFO-TAG, MSD) tagged goat polyclonal antibody (AF2449, R&D) was added and allowed to bind to the captured iduronate 2-sulfatase or chemically modified iduronate 2-sulfatase. The plate was washed and 2× Read Buffer (MSD) was added. The plate content was analyzed using a MSD Sector 2400 Imager Instrument. The instrument applies a voltage to the plate electrodes, and the SULFO-TAG label, bound to the electrode surface via the formed immune complex, will emit light. The instrument measures the intensity of the emitted light which is proportional to the amount of iduronate 2-sulfatase or chemically modified iduronate 2-sulfatase in the sample. The amount of iduronate 2-sulfatase or chemically modified iduronate 2-sulfatase was determined against a relevant iduronate 2-sulfatase or chemically modified iduronate 2-sulfatase standard.

### Results

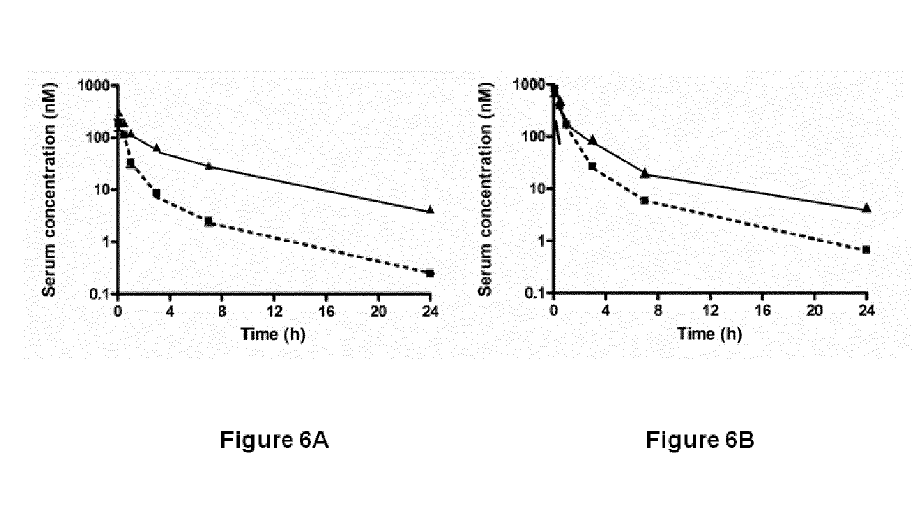

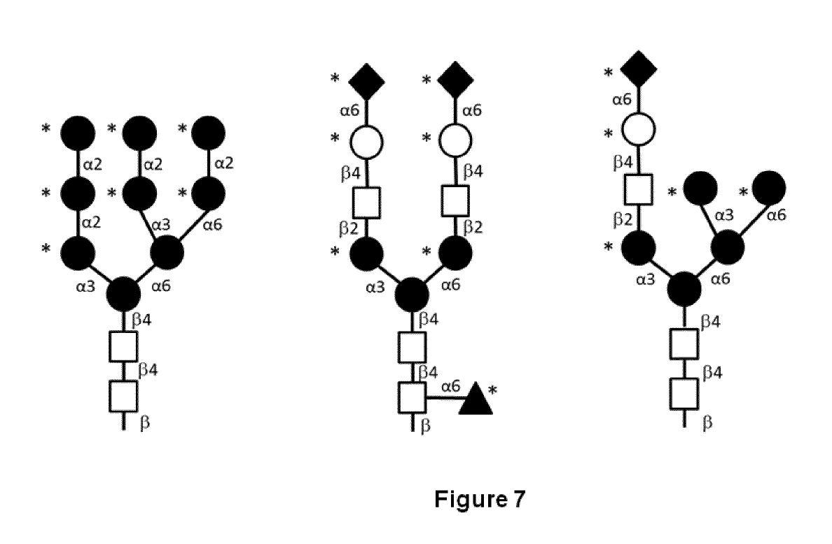

The serum clearance in mice of modified iduronate 2-sulfatase by method 2 was reduced 4-fold as compared to unmodified iduronate 2-sulfatase, see Table 1 below and FIG. 6. Whereas for iduronate 2-sulfatase modified according to method 3 it was reduced by 1.7 fold. Thus, both methods give a robust prolongation of serum half live of iduronate 2-sulfatase. This is probably at least partly due to the inhibition of receptor mediated uptake in peripheral tissue following chemical modification of iduronate 2-sulfatase.

TABLE 1
Serum clearance of iduronate 2-sulfatase
and modified iduronate 2-sulfatase

| | Dose | Serum CL |
|---|---|---|
| Test article | (mg/kg) | (mL/(h · kg)) |
| iduronate 2-sulfatase (SEQ ID NO: 1) | 1 | 60 |
| modified iduronate 2-sulfatase (New | 1 | 14 |
| method 2, SEQ ID NO: 1) | | |
| iduronate 2-sulfatase (SEQ ID NO: 1) | 3 | 50 |
| modified iduronate 2-sulfatase (New | 3 | 30 |
| method 3, SEQ ID NO: 1) | | |

### Example 7

### Potency of Modified Iduronate 2-Sulfatase on Glucosaminoglycan Storage in the Brain of a Living Animal

The usefulness of iduronate 2-sulfatase, produced according to the new methods described in Example 4, to treat neurological complications associated with MPS-II is evaluated in a mouse model of the disease in a manner similar to that described in Assunta-Polito et al, Hum Mol Genet. 19:4871-4885 (2010). This mouse is deficient in iduronate 2-sulfatase and shows cellular and pathological phenotypes similar to the human patients.

Modified iduronate 2-sulfatase is administered i.v., e.g. every other day for one month. A primary measure of the efficacy of the modified 2-sulfatase is the glucosaminoglycan levels in the brain of the mouse.

### Example 8

### Analysis of Glycan Structure after Chemical Modification of Sulfamidase According to Previously Known Method

In order to characterize the end product of chemical modification according to the previously known method, another sulfatase, namely sulfamidase (SEQ ID NO:2) was chemically modified according to the known method and characterized. Sulfamidase is due to its glycopeptide characteristics a suitable model protein for precise product identification after chemical modification.

### Material and Methods

### Chemical Modification According to the Known Method:

The chemical modification of sulfamidase according to the known method was performed as described in Example 1.

### Glycosylation Analysis:

The glycosylation pattern was determined for unmodified and different modified sulfamidase batches. Prior to glycopeptide analysis, sulfamidase (ca 10 μg) was reduced, alkylated and digested with trypsin. Reduction of the protein was done by incubation in 5 μl DTT 10 mM in 50 mM NH₄HCO₃ at 70° C. for 1 h. Subsequent alkylation with 5 μl iodoacetamide 55 mM in 50 mM NH₄HCO₃ was performed at room temperature (RT) and in darkness for 45 min. Lastly, the tryptic digestion was performed by addition of 30 μl of 50 mM NH₄HCO₃, 5 mM CaCl₂, pH 8, and 0.2 μg/μl trypsin in 50 mM acetic acid (protease: protein ratio 1:20 (w/w)). Digestion was allowed to take place over night at 37° C.

Five peptide fragments of the trypsin digested sulfamidase contained potential N-glycosylation sites. These peptide fragments containing potential glycosylation sites N(x), where x refers the position of the asparagine in the sulfamidase amino acid sequence as defined in SEQ ID NO:2, were:

N(21) containing fragment (residue 4-35 of SEQ ID NO:2, 3269.63 Da)
N(122) containing fragment (residue 105-130 of SEQ ID NO:2, 2910.38 Da)
N(131) containing fragment (residue 131-134 of SEQ ID NO:2, 502.29 Da)
N(244) containing fragment (residue 239-262 of SEQ ID NO:2, 2504.25 Da)
N(393) containing fragment (residue 374-394 of SEQ ID NO:2), 2542.22 Da
The molecular mass of each peptide fragment is given.

Possible glycosylation variants of the five tryptic peptide fragments were investigated by glycopeptide analysis. This was performed by liquid chromatography followed by mass spectrometry (LC-MS) on an Agilent 1200 HPLC system coupled to an Agilent 6510 Quadrupole time-of-flight mass spectrometer (Q-TOF-MS). Both systems were controlled by MassHunter Workstation. LC separation was performed by the use of a Waters XSELECT

CSH 130 C18 column (150×2.1 mm), the column temperature was set to 40° C. Mobile phase A consisted of 5% acetonitrile, 0.1% propionic acid, and 0.02% TFA, and mobile phase B consisted of 95% acetonitrile, 0.1% propionic acid, and 0.02% TFA. A gradient of from 0% to 10% B for 10 minutes, then from 10% to 70% B for another 25 min was used at a flow rate of 0.2 mL/min. The injection volume was 10 μl. The Q-TOF was operated in positive-electrospray ion mode. During the course of data acquisition, the fragmentor voltage, skimmer voltage, and octopole RF were set to 90, 65, and 650 V, respectively. Mass range was between 300 and 2800 m/z. Resulting modifications on the glycan moieties on four tryptic peptide fragments containing the N glycosylation sites N(21), N(131), N(244) and N(393) were investigated by LC-MS analysis.

### Results

### Glycosylation Analysis:

The type of glycosylation found on the four glycosylation sites prior to the chemical modification was predominantly complex glycans on N(21) and N(393), and oligomannose type of glycans on N(131) and N(244).

After the chemical modification, detailed characterization of the modified glycan structure was performed on the most abundant chemically modified glycopeptides (less abundant glycans were not detectable due to significant decrease in sensitivity as a result of increased heterogeneity of the glycans after chemical modification). In this Example, the modification on Man-6 glycan after chemical modification according to the known method is investigated.

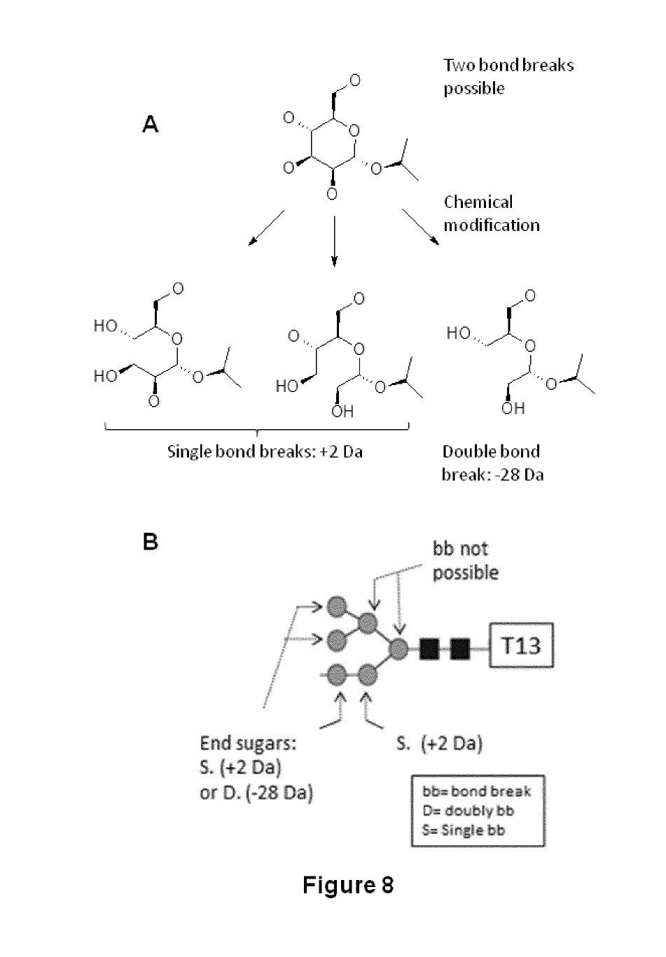

Periodate treatment of glycans cleaves carbon bonds between two adjacent hydroxyl groups of the carbohydrate moieties and alter the molecular mass of the glycan chain. FIG. 8A illustrates an example of predicted bond breaks on mannose after chemical modification. FIG. 8B depicts a model of Man-6 glycan showing the theoretical bond breaks that may take place after oxidation with sodium periodate.

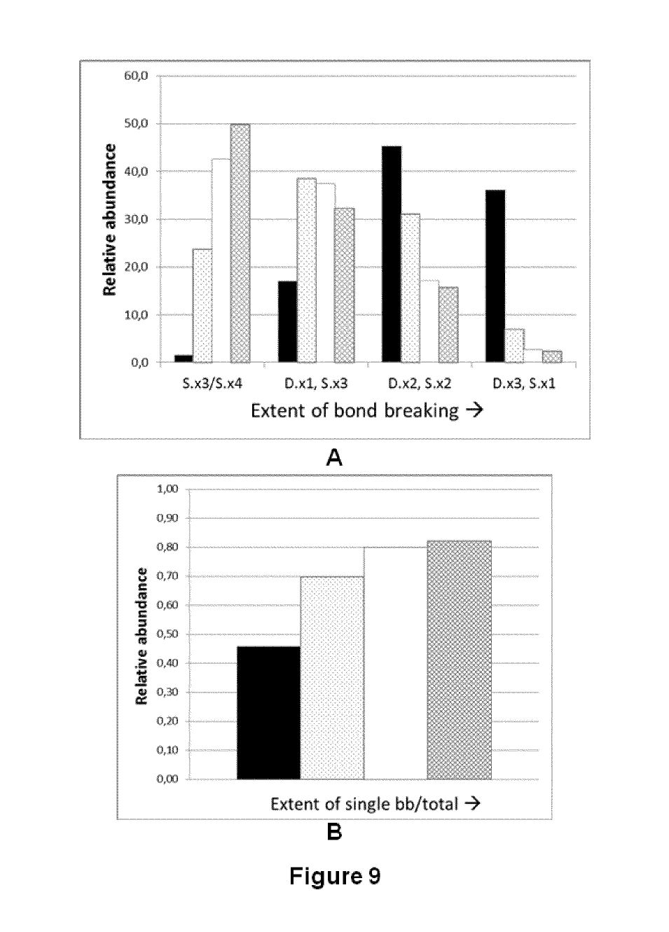

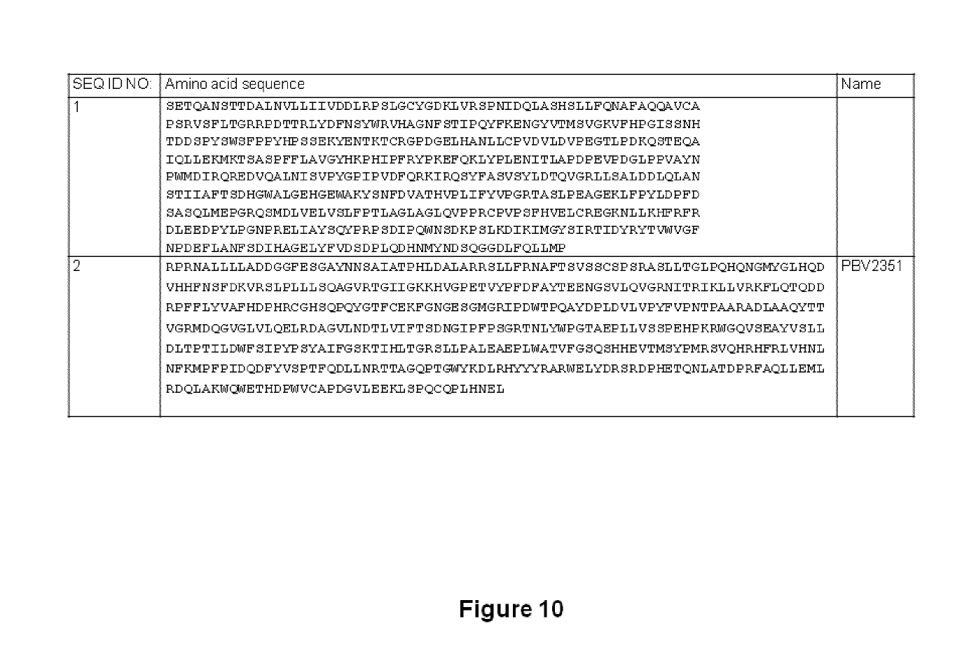

The tryptic peptide NITR with Man-6 glycan attached to N(131) (T13+Man-6 glycan) was analyzed by mass spectrometry, prior to and after chemical modification according to the previously known method (results not shown). Ions corresponding to the chemically modified glycopeptide with various degree of bond breaking were identified. For Man-6 glycan, there can theoretically be a maximum of 3 double bond breaks and one single bond break. When the modification was performed according to the known method, the most intense ion signal in the mass spectrum was found to be corresponding to 2 double bond breaks and 2 single bond breaks, while the second most intense ion signal corresponded to 3 double bond breaks and one single bond break, which is the most extensive bond breaks possible. A diagram visualizing the extent of bond breaking found on T13+Man-6 glycan after chemical modification according to the known method is shown in FIG. 9 (due to isotopic distribution from the ions observed, the results are approximate but comparable).

The reproducibility of the chemical modification was tested by using three different batches of chemically modified sulfamidase produced according to the previously known method. The ions corresponding to different degree of bond breaking showed very similar distribution in the MS spectra from the three different batches.

### Example 9

### Analysis of Glycan Structure after Chemical Modification of Sulfamidase According to New Methods 1, 2, and 3

### Material and Methods

Sulfamidase, another sulfatase, was chemically modified according to the following new methods.

### New Method 1:

Sulfamidase produced in Quattromed Cell Factory (QMCF) episomal expression system (Icosagen AS), was oxidized by incubation with 20 mM sodium meta-periodate at 0° C. in the dark for 120 min in phosphate buffers having a pH of 6.0. Glycan oxidation was quenched by addition of ethylene glycol to a final concentration of 192 mM. Quenching was allowed to proceed for 15 min at 6° C. before sodium borohydride was added to the reaction mixture to a final concentration of 50 mM. After incubation at 0° C. for 120 min in the dark, the resulting sulfamidase preparation was ultrafiltrated against 20 mM sodium phosphate, 100 mM NaCl, pH 6.0.

### New Method 2:

Sulfamidase was oxidized by incubation with 10 mM sodium meta-periodate at 0° C. in the dark for 180 min in acetate buffer having an initial pH of between 4.5 to 5.7. Glycan oxidation was quenched by addition of ethylene glycol to a final concentration of 192 mM. Quenching was allowed to proceed for 15 min at 6° C. before sodium borohydride was added to the reaction mixture to a final concentration of 25 mM. After incubation at 0° C. for 60 min in the dark, the resulting sulfamidase preparation was ultrafiltrated against 10 mM sodium phosphate, 100 mM NaCl, pH 7.4.

### New Method 3:

Sulfamidase produced in a stable cell line according to Example 1 was oxidized by incubation with 10 mM sodium meta-periodate at 8° C. in the dark for 60 min in acetate buffer having an initial pH of 4.5. Glycan oxidation was quenched by addition of ethylene glycol to a final concentration of 192 mM. Quenching was allowed to proceed for 15 min at 6° C. before sodium borohydride was added to the reaction mixture to a final concentration of 25 mM. After incubation at 0° C. for 60 min in the dark, the resulting sulfamidase preparation was ultrafiltrated against 10 mM sodium phosphate, 100 mM NaCl, pH 7.4.

### Glycosylation Analysis:

The glycosylation analysis was performed according to the LC-MS method described in Example 8. Resulting modifications on the glycan variants of the four tryptic peptide fragments containing the N glycosylation sites N(21), N(131), N(244) and N(393) were investigated by LC-MS analysis.

### Results

### Glycosylation Analysis:

Detailed characterization of the modified glycan profile on sulfamidase, chemically modified according to new methods 1, 2, and 3, was performed on the most abundant chemically modified glycopeptides. In this Example, the modification on Man-6 glycan after chemical modification according to new methods 1, 2, and 3, was investigated.

Ions corresponding to the chemically modified glycopeptide T13+Man-6 glycan with various degree of bond breaking were identified. Theoretically there can be a maximum of 3 double bond breaks and one single bond break (see FIG. 8B a model of Man-6 glycan showing the bond breaks possible to occur after oxidation with sodium periodate). When the modification was performed according to the new method 1, the most intense ion signal in the mass spectrum (not shown) was found to be corresponding to one double bond break and 3 single bond breaks, while the second most intense ion signal corresponded to 2 double bond breaks and 2 single bond breaks. When the modification was performed according to new methods 2 and 3, the bond breaks on Man-6 glycan were even further shifted to preferentially single bond breaks. In FIG. 9A is shown a diagram visualizing the extent of bond breaking of the tryptic peptide T13+Man-6 glycan after chemical modification.

The reproducibility of the chemical modification was tested by using triplicates (new method 1) or duplicates (new methods 2) of chemically modified sulfamidase.

When comparing the Man-6 glycan modifications resulting from sulfamidase chemically modified according to the known method with the Man-6 glycan modifications resulting from sulfamidase chemically modified according to the new methods 1, 2, and 3, there was a large difference in degree of bond breaking. This is illustrated in FIG. 9A, where the distribution of the different degrees of bond breaking is plotted for the four methods (due to isotopic distribution from the ions observed, the results are approximate, but comparable).

FIG. 9B shows the relative abundance of single bond breaks for the methods used. The previously known method provides a modified sulfamidase having 45% single bond breaks in the investigated Man-6-glycan, while the new methods 1, 2, and 3 have 70, 80, and 82% single bond breaks, respectively, after chemical modification.

Subsequently, the milder methods of chemical modification described herein provides a product with significantly less double bond breaks in modified glycans.

### Example 10

### Analysis of Glycan Structure after Chemical Modification of Iduronate 2-Sulfatase According to Previously Known Method

As evident from SDS-PAGE analysis (FIG. 2A), chemical modification according to the previously known method resulted in the lowering of the molecular weight of iduronate 2-sulfatase. In order to characterize iduronate 2-sulfatase modified according to the previously known method, glycosylation analysis is performed according to the methods described in Example 8.

### Material and Methods

### Chemical Modification According to the Known Method:

The chemical modification of sulfamidase according to the known method is performed as described in Example 1.

### Glycosylation Analysis:

The glycosylation analysis is performed according to the LC-MS method described in Example 2 and 8.

### Results

Chemical modification of iduronate 2-sulfatase according to the known method is expected to give rise to modified glycopeptides(s) for which the extent of bond breaking is similar to the extent of bond breaking in glycopeptides of modified sulfamidase (see Example 8). Thus, the known method is expected to give rise to predominantly double bond breaks in the glycan moieties, which could explain the loss of molecular weight observed on SDS-PAGE (FIG. 2A, lane 2 vs. 3).

### Example 11

### Analysis of Glycan Structure after Chemical Modification of Iduronate 2 Sulfatase According to New Methods 1, 2, and 3

### Material and Methods

Iduronate 2-sulfatase modified according to the methods described in Example 3 is subjected to glycosylation analysis.

### Glycosylation Analysis:

The glycosylation analysis is performed according to the LC-MS method described in Example 10.

### Results

The new methods of chemical modification described herein provides a product with significantly less double bond breaks in modified glycans, which is also reflected in the difference in molecular weight of iduronate 2-sulfatase produced by the different methods (FIG. 2B).

### Example 12

### Chemical Modification of Iduronate 2-Sulfatase in the Presence of an Active Site Protecting Ligand

As described in Example 3 new method 6, oxidation (step a)) can be performed in the presence of a ligand. The ligand can be a substrate as exemplified by 4-methylumbeliferone iduronide-sulfate. Alternatively, any other known ligand of iduronate 2-sulfatase, such as sulfate, can be used. Heparin or heparin sulfate of any origin could also be used as an additive throughout one or more of the reaction steps.

### Example 13

### Chemical Modification of Iduronate 2-Sulfatase Immobilized on a Gel Matrix

The modification method as described herein, and in particular, new method 1-6 of Example 3, is performed while iduronate 2-sulfatase is immobilized on a gel matrix. By using a POROS® XQ Strong Anion Exchange column, iduronate 2-sulfatase is immobilized by loading the column using a sodium phosphate buffer with a pH of 7.5. Following loading of iduronate 2-sulfatase, the column is equilibrated with solutions for step a), quenching of step a), step b), and quenching of step b) in a consecutive fashion. Elution of chemically modified iduronate 2-sulfatase is performed by washing the column with a buffer containing 100 mM sodium phosphate and 150 mM sodium chloride with a pH of 5.6.

### Example 14

### Chemical Modification of Iduronate 2-Sulfatase in a Continuous Process

The modification method as described herein, and in particular, new methods 1-6 of Example 3, is performed in a continuous mode. By applying a continuous flow, e.g. by utilizing a HPLC pump or similar equipment, a solution of iduronate 2-sulfatase is transported through a tubing. The tubing can be of any inert material, e.g. ethylenetetrafluoroethylene or polytetrafluoroethylene. By adjusting the speed of flow and the inner tubing diameter, speed of transport within the system is precisely adjusted. By applying inlets at defined positions, stock solutions of the reagents of the chemical modification are added in a continuous mode. This can be achieved in a multi-pump HPLC system, e.g. an Akta avant (GE Healthcare). At each point of inlet a small-volume mixing chamber is added, similar to those present in most multi-pump HPLC systems.

In a specific continuous mode example of the new methods 1-6, reagents are added at an inlet (valve) at a flowrate that is approximately 10 of that for the iduronate 2-sulfatase solution. Stock solution of reagents are prepared at a concentration that is ten-fold higher the concentration accounted for in new method 1-6 in Example 3.

### Example 15

### Distribution of Modified Iduronate 2-Sulfatase to Brain of Iduronate 2-Sulfatase Deficient Mice

### Materials and Methods

The distribution of intravenously (iv) administrated modified iduronate 2-sulfatase produced according to new method 2 of Example 3 to brain in vivo was investigated.

### Test Article Preparation:

Modified iduronate 2-sulfatase was formulated at 2 mg/mL, sterile filtrated and frozen at −70° C. until used.

### Animals:

Male mice, IDS-KO (B6N.Cg-Idstm1Muen/J)(Jackson Laboratories, ME, USA), were used. The animals were housed singly in cages at 23±1° C. and 40-60% humidity, and had free access to water and standard laboratory chow. The 12/12 h light/dark cycle was set to lights on at 7 pm. The animals were conditioned for at least two weeks before initiating the study. The mice were given an intravenous administration in the tail vein of 10 mg/kg modified iduronate 2-sulfatase. The study was finished 24 h after the last injection. The mice were anaesthetized by isoflurane. Blood was withdrawn from retro-orbital plexus bleeding. Perfusion followed by flushing 20 mL saline through the left ventricle of the heart. Brain was dissected weighed and frozen rapidly in liquid nitrogen. Brain homogenates where prepared and activity was assessed using the method described in example 2 with addition of 10 mM lead acetate in the assay buffer as adjustment to the protocoll.

### Results

Activity of modified iduronate 2-sulfatase in perfused brain homogenates of IDS-KO mice could be confirmed. An average activity of 1.8±0.4 μM/min (n=4) was determined under the assay conditions used.

1. A modified iduronate 2-sulfatase comprising substantially no epitopes for glycan recognition receptors, wherein said iduronate 2-sulfatase has a catalytic activity of at least 50% of that of unmodified iduronate 2-sulfatase in vitro.

2. A modified iduronate 2-sulfatase according to claim 1, wherein said iduronate 2-sulfatase has catalytic activity in the brain of said mammal.

3. A modified iduronate 2-sulfatase according to claim 1, wherein said iduronate 2-sulfatase has catalytic activity in peripheral tissue.

4. A modified iduronate 2-sulfatase according to claim 3, wherein said peripheral tissue is joints, bone, connective tissue and/or cartilage.

5. The modified iduronate 2-sulfatase according to claim 1, having a relative content of natural glycan moieties being around 38% or less of the content of natural glycan moieties in unmodified recombinant iduronate 2-sulfatase.

6. The modified iduronate 2-sulfatase according to claim 1, wherein natural glycan moieties of said iduronate 2-sulfatase are disrupted by single bond breaks and double bond breaks, the extent of single bond breaks being at least 60% in oligomannose glycans.

7. The modified iduronate 2-sulfatase according to claim 1, comprising a polypeptide consisting of an amino acid sequence as defined in SEQ ID NO:1, or a polypeptide having at least 90% sequence identity with an amino acid sequence as defined in SEQ ID NO:1, wherein said epitopes are absent at at least five of the eight N-glycosylation sites: asparagine (N) in position 6 (N(6)), asparagine (N) in position 90 (N(90)), N in position 119 (N(119)), N in position 221 (N(221)), N in position 255 (N(255)), N in position 300 (N(300)), N in position 488 (N(488)) and N in position 512 (N(512)) of SEQ ID NO:1.

8. The modified iduronate 2-sulfatase according to claim 7, wherein the epitope at the glycosylation site asparagine (N) in position 90 (N(90)) is absent.

9. The modified iduronate 2-sulfatase according to claim 7, wherein said epitopes are absent at all of said eight N-glycosylation sites.

10. The modified iduronate 2-sulfatase according to claim 7, comprising a Cα-formylglycine residue in position 59 of SEQ ID NO:1 (FGly59) providing said catalytic activity.

11. An iduronate 2-sulfatase composition, comprising modified iduronate 2-sulfatase according to claim 1, said composition having a Ca-formylglycine (FGly) to serine (Ser) ratio at the active site that is greater than 1.

12. A method of preparing a modified iduronate 2-sulfatase, said method comprising:
a) reacting a glycosylated iduronate 2-sulfatase with an alkali metal periodate, and
b) reacting said iduronate 2-sulfatase with an alkali metal borohydride for a time period of no more than 2 h;
thereby modifying glycan moieties of the iduronate 2-sulfatase and reducing the activity of the iduronate 2-sulfatase with respect to glycan recognition receptors, while retaining at least 50% catalytic activity of said iduronate 2-sulfatase in vitro.

13. The method according to 12, wherein step a) and step b) are performed in sequence without performing dialysis, ultrafiltration, precipitation or buffer exchange.

14. The method according to claim 12, wherein step a) is further characterized by at least one of:
i) said alkali metal periodate is sodium meta-periodate;
ii) said periodate is used at a concentration of no more than 20 mM;
iii) said reaction is performed at a temperature of between 0 and 22° C.;
iv) said reaction is performed for a time period of no more than 4 h; and
v) said reaction of step a) is performed at a pH of 3-7.

15. The method according to claim 12, wherein step b) is further characterized by at least one of:
i) said alkali metal borohydride is sodium borohydride;
ii) said borohydride is used at a concentration of no more than 4 times the concentration of said periodate;
iii) said reaction is performed for a time period of no more than 1.5 h; and
iv) said reaction is performed at a temperature of between 0 and 8° C.

16. The method according to claim 12, wherein step a) is performed for a time period of no more than 3 h and step b) is performed for no more than 1 h, and said borohydride optionally is used at a concentration of no more than 4 times the concentration of said periodate.

17. The method according to claim 12, further comprising a2) quenching of the reaction resulting from step a); and/or b2) quenching of the reaction resulting from step b).

18. The method according to claim 12, wherein the active site of said iduronate 2-sulfatase is made inaccessible to oxidative and/or reductive reactions during at least one of steps a) and b).

19. The method according to claim 18, wherein at least one of steps a) and b) of the method is/are performed in the presence of a protective ligand.

20. The method according to claim 18, wherein steps a) and b) of the method are performed while iduronate 2-sulfatase is immobilized on a resin.

21. A modified iduronate 2-sulfatase obtainable by the method according to claim 12.

22. (canceled)

23. (canceled)

24. A method of treating a mammal afflicted with a lysosomal storage disease, comprising administering to the mammal a therapeutically effective amount of a modified iduronate 2-sulfatase according to claim 1.


SEQUENCE LISTING (2 sequences)

SEQ ID NO: 1 - protein, 525 aa, HOMO SAPIENS
SETQANSTTDALNVLLIIVDDLRPSLGCYGDKLVRSPNIDQLASHSLLFQNAFAQQAVCAPSRVSFLTGRRPDTTRLYDFNSYWRVHAGNFSTIPQYFKENGYVTMSVGKVFHPGISSNHTDDSPYSWSFPPYHPSSEKYENTKTCRGPDGELHANLLCPVDVLDVPEGTLPDKQSTEQAIQLLEKMKTSASPFFLAVGYHKPHIPFRYPKEFQKLYPLENITLAPDPEVPDGLPPVAYNPWMDIRQREDVQALNISVPYGPIPVDFQRKIRQSYFASVSYLDTQVGRLLSALDDLQLANSTIIAFTSDHGWALGEHGEWAKYSNFDVATHVPLIFYVPGRTASLPEAGEKLFPYLDPFDSASQLMEPGRQSMDLVELVSLFPTLAGLAGLQVPPRCPVPSFHVELCREGKNLLKHFRFRDLEEDPYLPGNPRELIAYSQYPRPSDIPQWNSDKPSLKDIKIMGYSIRTIDYRYTVWVGFNPDEFLANFSDIHAGELYFVDSDPLQDHNMYNDSQGGDLFQLLMP

SEQ ID NO: 2 - protein, 482 aa, HOMO SAPIENS
RPRNALLLLADDGGFESGAYNNSAIATPHLDALARRSLLFRNAFTSVSSCSPSRASLLTGLPQHQNGMYGLHQDVHHFNSFDKVRSLPLLLSQAGVRTGIIGKKHVGPETVYPFDFAYTEENGSVLQVGRNITRIKLLVRKFLQTQDDRPFFLYVAFHDPHRCGHSQPQYGTFCEKFGNGESGMGRIPDWTPQAYDPLDVLVPYFVPNTPAARADLAAQYTTVGRMDQGVGLVLQELRDAGVLNDTLVIFTSDNGIPFPSGRTNLYWPGTAEPLLVSSPEHPKRWGQVSEAYVSLLDLTPTILDWFSIPYPSYAIFGSKTIHLTGRSLLPALEAEPLWATVFGSQSHHEVTMSYPMRSVQHRHFRLVHNLNFKMPFPIDQDFYVSPTFQDLLNRTTAGQPTGWYKDLRHYYYRARWELYDRSRDPHETQNLATDPRFAQLLEMLRDQLAKWQWETHDPWVCAPDGVLEEKLSPQCQPLHNEL